(19) United States
(12) Patent Application Publication
(10) Pub. No.: US 2026/0108181 A1
(43) Pub. Date: Apr. 23, 2026

(54) IN-EAR SYSTEM FOR MONITORING LOWER JAW MOTION

(71) Applicant: UNIVERSITY HEALTH NETWORK, Toronto (CA)

(72) Inventors: Nevena MUSIKIC, Miami, FL (US); Milos R. POPOVIC, Mississauga (CA)

(21) Appl. No.: 19/361,658

(22) Filed: Oct. 17, 2025

Related U.S. Application Data
(60) Provisional application No. 63/708,820, filed on Oct. 18, 2024.

(51) Int. Cl. A61B 5/11; A61B 5/00

(52) U.S. Cl. CPC A61B 5/1123; A61B 5/0017; A61B 5/0086; A61B 5/1126; A61B 5/6817; A61B 5/7264

(57) ABSTRACT

Systems, methods, and devices for detecting mandibular motion via in-ear monitoring of ear canal deformation are disclosed. An earpiece houses multiple infrared proximity sensors oriented to measure volumetric changes in the cartilaginous portion of the outer ear canal induced by jaw movement. A coupled processor acquires, filters, and fuses proximity signals to identify activity onset and termination, and classifies movement events such as chewing, coughing, and yawning using signal-processing and machine-learning algorithms. The system optionally communicates wirelessly to a voice transducer to enable hands-free voice initiation and control. The system provides discreet, reproducible, and user-specific operations suitable for implementing a hands-free human-machine interface.

### CROSS-REFERENCE TO RELATED APPLICATION

This application claims all benefit including priority to U.S. Provisional Patent Application No. 63/708,820 filed on Oct. 18, 2024 entitled “IN-EAR SYSTEM FOR MONITORING LOWER JAW MOTION”, the entire contents of which is hereby incorporated by reference.

### FIELD

This disclosure relates to monitoring lower jaw motion, and more specifically relates to monitoring lower jaw motion using an in-ear system.

### BACKGROUND

Laryngectomy patients, who have undergone larynx removal as part of laryngeal cancer treatment, experience disruption in breathing, swallowing, and speaking functions due to the larynx's vital role in these processes. Following laryngectomy, a tracheostomy is created to establish an airway (breathing pathway) that connects the lungs to the external environment. Laryngectomy results in the loss of sound generation; however, articulation and shaping of sound for speech is retained.

Two primary methods for speech rehabilitation post-laryngectomy are the tracheoesophageal puncture prosthesis (TEP) and the electrolarynx (EL). TEP is a one-way valve that is inserted through the tracheal-esophageal common wall, redirecting airflow from the lungs into the esophagus and pharynx, enabling sound generation through pharyngeal vibration. EL, an electrical alternative to TEP, is a handheld device that is placed on the neck and transmits vibrations into the pharynx (throat) to be shaped by the articulatory organs. Both TEP and EL present a common challenge, requiring at least one hand, to occlude the tracheostomy or hold the electrolarynx.

The use of a hand to produce speech limits activities of daily living, work and recreational activities. The absence of effective, hands-free speech restoration methods post-laryngectomy hinders patients' ability to perform simple, dual-task activities, further exacerbating the challenges they face, even leading to isolation and depression.

There have been attempts to achieve hands-free voice control for laryngectomy patients, but no breakthrough technology has been developed. These include surface electromyography (sEMG) signals, pneumatic technology utilizing pressure measurements at the stoma, and video technology. Common challenges across these solutions include their public visibility, difficulty with use in a non-laboratory setting, the need for patient training and the cognitive load required to operate the device.

Thus, there is a need for improved or alternative solutions that address one or more of the limitations of existing solutions.

### SUMMARY

In accordance with an aspect, there is provided a system for monitoring lower jaw motion. The system includes an insert for insertion into an ear canal of a user, the insert including a plurality of optical sensors, each for sensing deformation of a corresponding region of the ear canal caused by a lower jaw motion; and one or more processors and one or more memories coupled with the one or more processors, the processors and memories configured to: receive a deformation signal from at least one of the optical sensors, the deformation signal indicative of deformation of the corresponding region of the ear canal; and process the deformation signal to generate an output signal corresponding to the lower jaw motion.

In such system, the plurality of optical sensors may include an infrared proximity sensor.

In such system, the plurality of optical sensors may include an optical sensor aligned to sense deformation of an anterior wall of the ear canal.

In such system, the plurality of optical sensors may include an optical sensor aligned to sense deformation of a posterior wall of the ear canal.

In such system, the plurality of optical sensors may include an optical sensor aligned to sense deformation of an inferior cartilaginous wall of the ear canal.

In such system, the plurality of optical sensors may include an optical sensor aligned towards an eardrum.

The system may further include a wireless transceiver to transmit the deformation signal wirelessly from the at least one of the optical sensors.

In such system, the insert may be a first insert for insertion into a first ear canal of the user, and the system may further include: a second insert for insertion into a second ear canal of the user, the second insert including a further plurality of optical sensors, each for sensing deformation of a corresponding region of the second ear canal caused by lower jaw motion.

In such system, the output signal may encode a particular user intent.

In such system, the particular user intent may include at least one of a speech intent, a chewing intent, or a swallowing intent.

In such system, the output signal may encode a particular user activity, request, or demand.

In such system, the particular user activity may include at least one of a particular unit of speech, a particular unit of chewing, a particular unit of smiling, a particular unit of laughing, a particular unit of yawning, or a particular unit of swallowing.

In such system, the particular user activity may include a particular kissing.

In such system, the output signal may be generated by applying a machine learning model to the deformation signal.

In accordance with another aspect, there is provided an in-ear device for monitoring lower jaw motion. The device includes an insert for insertion into an ear canal, the insert including a plurality of optical sensors, each for sensing deformation of a corresponding region of the ear canal caused by a lower jaw motion; and an output interface for transmitting a deformation signal sensed by at least one of the optical sensors, the deformation signal indicative of deformation of the corresponding region of the ear canal.

In such device, the plurality of optical sensors may include an infrared proximity sensor.

In such device, the plurality of optical sensors may include an optical sensor aligned to sense deformation of an anterior wall of the ear canal, a posterior wall of the ear canal, or an inferior cartilaginous wall of the ear canal.

In such device, the plurality of optical sensors may include an optical sensor aligned towards an eardrum.

In such device, the output interface may include a wireless transceiver to transmit the deformation signal wirelessly from the at least one of the optical sensors.

In accordance with a further aspect, there is provided a method for monitoring lower jaw motion including: receiving a deformation signal from at least one optical sensor for sensing deformation of a corresponding region of the ear canal caused by a lower jaw motion, disposed into an ear canal of a user; and processing the deformation signal to generate an output signal corresponding to the lower jaw motion.

Many further features and combinations thereof concerning embodiments described herein will appear to those skilled in the art following a reading of the instant disclosure.

### BRIEF DESCRIPTION OF THE DRAWINGS

In the figures,

FIG. 1 is a schematic diagram of a monitoring system for lower jaw motion, in accordance with an embodiment;

FIG. 2 depicts the anatomy of the outer ear canal with surrounding structures;

FIG. 3A and FIG. 3B show an in-ear device of a monitoring system, in accordance with an embodiment;

FIG. 4A, FIG. 4B, FIG. 4C, and FIG. 4D illustrate the positioning and orientation of optical sensors in a monitoring system, in accordance with an embodiment;

FIG. 5A shows a sensor coverage area of an optical sensor, in accordance with an embodiment;

FIG. 5B shows an optical sensor mounted on a sensor printed circuit board, in accordance with an embodiment;

FIG. 6 is a schematic diagram of a processing device of a monitoring system for lower jaw motion, in accordance with an embodiment;

FIG. 7 shows a master PCB of a processing device of a monitoring system for lower jaw motion, in accordance with an embodiment;

FIG. 8 shows example signals for fundamental mandibular movements, in accordance with an embodiment;

FIG. 9 shows a cross comparison of magnitude of change in the right and left ear canals during fundamental mandibular movements, in accordance with an embodiment;

FIG. 10 shows a comparison between proximity measurements in the outer ear canal and sEMG recordings during mastication and coughing, in accordance with an embodiment;

FIG. 11A shows results of the Assessment of Intelligibility of Dysarthric Speech (AIDS) test; FIG. 11B shows vocalization during sustained vowel production; and FIG. 11C shows an example of detection of declarative and interrogative phrases, in accordance with an embodiment;

FIG. 12 shows a comparison of onset and termination times detected in the proximity data with those of the sound envelope (Panel A); how a monitoring system can replicate signals for prompts corresponding to declarative, interrogative, imperative, and exclamatory sentences (Panel B); speech material used for the analysis of onset/termination times and reproducibility (Panel C), in accordance with an embodiment;

FIG. 13 shows fundamental mandibular movements (Panel A); integrated mandibular movements (Panel B); and mandibular speech dynamics (Panel C), in accordance with an embodiment;

FIG. 14 depicts experiment dataset structure, in accordance with an embodiment;

FIG. 15 shows spectrograms of proximity data during chewing, speaking, and a baseline measurement (left panel); and an amplitude spectrum of the baseline signal recorded in the ear canal (right panel), in accordance with an embodiment;

FIG. 16 shows a comparison between manual segmentation and sound-based segmentation of proximity data, in accordance with an embodiment;

FIG. 17 shows normalized sound and proximity signals illustrating pre-phonatory and post-phonatory activation in the proximity data, in accordance with an embodiment;

FIG. 18 shows feature refinement analysis, including correlation analysis, significance levels and Least Absolute Shrinkage and Selection Operator (LASSO) coefficients, in accordance with an embodiment;

FIG. 19 shows correlations between features after feature refinement, in accordance with an embodiment;

FIG. 20 shows predominant features and their distribution among subjects, in accordance with an embodiment;

FIG. 21A shows accuracy curves illustrating the relationship between the number of features and accuracy for each subject, in accordance with an embodiment;

FIG. 21B shows average accuracy with standard deviation across subjects with respect to the number of features used, in accordance with an embodiment;

FIG. 22 shows results of Leave-One-Patient-Out (LOPO) validation for the Boosted Trees classifier, in accordance with an embodiment;

FIG. 23A, FIG. 23B, FIG. 23C, and FIG. 23D show clusters of integrated mandibular movements measured at each ear canal, obtained through the k-means clustering, in accordance with an embodiment;

FIG. 24 shows outcomes of k-means clustering, in accordance with an embodiment;

FIG. 25 shows the wide neural network, featuring a single fully connected hidden layer, used for performance analysis (Panel A); and a Rectified Linear Unit (ReLU) activation function (Panel B), in accordance with an embodiment;

FIG. 26 shows results of evaluation of wide neural networks performance, in accordance with an embodiment;

FIG. 27 shows classification results, in accordance with an embodiment;

FIG. 28 shows results of correlation analysis of the first three principal components in the data, in accordance with an embodiment;

FIG. 29 is a schematic diagram of a computing device, in accordance with an embodiment; and

FIG. 30 is a process diagram illustrating the method employed by the system to monitor lower jaw movement using ear canal deformation signal, in accordance with an embodiment.

These drawings depict exemplary embodiments for illustrative purposes, and variations, alternative configurations, alternative components and modifications may be made to these exemplary embodiments.

### DETAILED DESCRIPTION

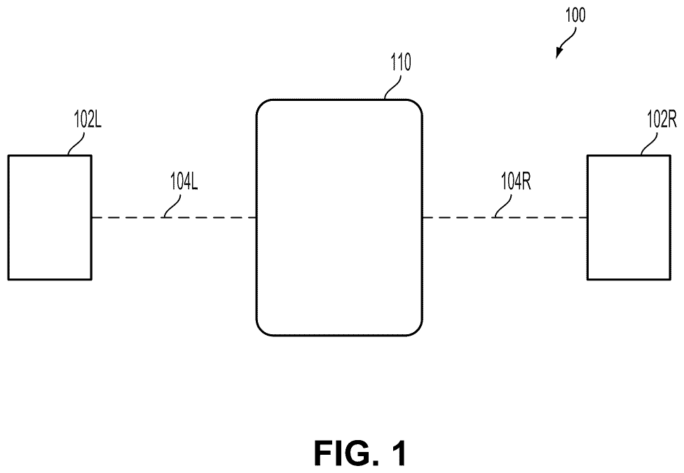

FIG. 1 is a schematic diagram of a monitoring system 100 for monitoring lower jaw motion of a user, in accordance with an embodiment. Monitoring system 100 is configured to monitor lower jaw motion based on volumetric change in cartilaginous parts of the user's outer ear canal(s).

As detailed herein, embodiments of monitoring system 100 may be configured to monitor a diverse range of lower jaw movements. Such movements may indicate the occurrence of specific activities such as speech, chewing, speaking, yawning, and other orofacial activities.

Monitoring system 100 includes a pair of in-ear devices 102 suitable for insertion into the user's respective left and right ear canals. In-ear device 102 for insertion into the left ear canal may be referred to as in-ear device 102L, while in-ear device 102 for insertion into the right ear canal may be referred to as in-ear device 102R.

Each in-ear devices 102 includes one or more optical sensors, each configured to sense deformation of a corresponding region of an ear canal caused by a lower jaw motion. Each in-ear device 102 is communicatively coupled to a processing device 110 and communicates sensor signal data to processing device 110 for processing. Such processing at processing device 100 may be used to detect, from the sensed signal data, an activity of the user. Such activity may include, for example, a particular intent such as a speech intent, a chewing intent, a swallowing intent, or the like. Detection of such activity may include detecting a particular unit of speech, a particular unit of chewing, a particular unit of smiling, a particular unit of laughing, a particular unit of yawning, a particular unit of swallowing, or the like. Such activity may also include, for example, kissing, or other orofacial activities involving movement of the lower jaw.

In some embodiments, processing at processing device 110 may be used to detect, from the sensed signal data, a particular user request or command. In such embodiments, monitoring system 100 may be used to provide an interface for human-computer interaction, where jaw movements are used as a form of user input, e.g., for artificial speech control, computer user interface control, or the like.

In some embodiments, processing device 110 is sized and shaped as a bracelet or necklace, or other form wearable by the user.

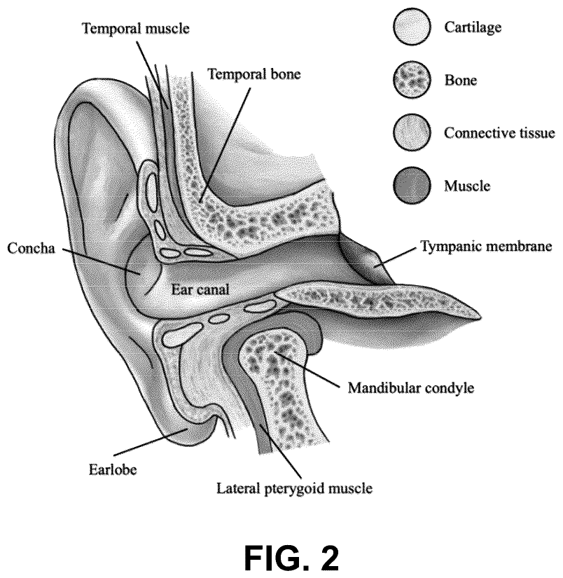

FIG. 2 depicts anatomy of an outer ear canal with surrounding structures. The ear canal consists of two parts, the outer (soft and pliable) and inner canal (rigid and bony). The soft part, not directly attached to the lower jaw, is surrounded by highly deformable tissue (skin, fat, and cartilage). When the lower jaw is moved forward, a small void is created due to the sliding of the mandibular condyle and deformable tissue fills the void causing the canal to deform and change its volume. This change in volume serves as a clear indicator of lower jaw movement. The anterior wall of the cartilaginous ear canal undergoes the most noticeable deformation with movement of the mandible making it a location of interest for signal detection.

The change in ear canal volume with different lower jaw positions is most prominently observed in the anterior-posterior (A-P) plane, while the superior-inferior (S-I) plane remains relatively unaffected.

Conveniently, in some embodiments, disposing in-ear devices 102 within an ear canal may reduce interference with daily activities, and their portability and small form factor allow for prolonged wear throughout the day.

Conveniently, in some embodiments, disposing in-ear devices 102 within the ear canal provides shielding from external influences such as, e.g., environmental noise, light, or electromagnetic interference.

In the depicted embodiment, monitoring system 100 includes two in-ear devices 102. However, in some embodiments, monitoring system 100 includes only one in-ear device 102, and sensor data is obtained from only one ear canal.

According to experimental data, the magnitude of volume change differs between the left and right ear, and the direction of change can be either positive or negative. Based on the symmetry of magnitude and direction, four distinct categories have been identified. The most prevalent category, observed in over 50% of subjects, exhibits asymmetric magnitude between the left and right ear but symmetric direction of change (positive or negative change in both ears). Thus, in embodiments with only one in-ear devices 102, such device 102 should be selected for the ear that maximizes the magnitude of change for a particular user.

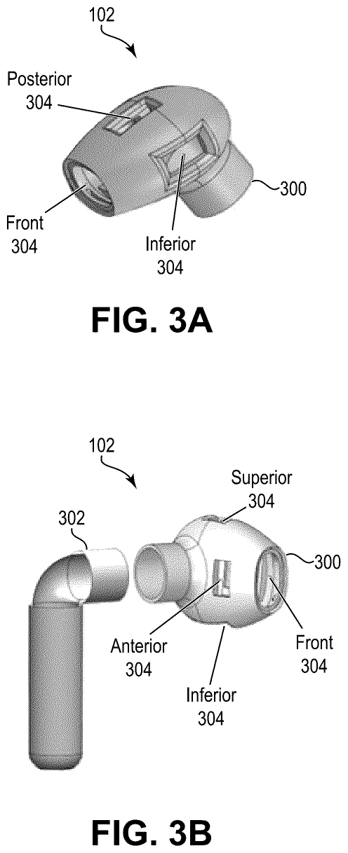

As depicted in FIG. 3A and FIG. 3B, in-ear device 102 includes a sensor assembly 300 and a handle assembly 302 joined thereto. Sensor assembly 300 includes one or more optical sensors, each for sensing deformation of a corresponding region of an ear canal caused by lower jaw motion. Handle assembly 302 provides a handle to allow a user to manipulate in-ear device 102 (e.g., for insertion and removal). Handle assembly 302 includes a power source (e.g., a lithium battery or other power source) and an output interface for wired or wireless communication. For example, the output interface may be an I2C connector. For example, the output interface may be a Bluetooth transceiver.

As depicted, sensor assembly 300 includes a portion sized and shaped for insertion into an ear canal and thereby dispose optical sensors at a location suitable for sensing regions of the ear canal. In some embodiments, this portion is sized and shaped to resemble a conventional earbud (e.g., a consumer device for listening to music). In some embodiments, this portion is tailored to the length and diameter of a particular user's ear canal. In some embodiments, the size and shape of this portion may be selected to take into consideration its intended use throughout the day, necessitating a lightweight and comfortable design.

As depicted, the housing of sensor assembly 300 includes a plurality of openings 304 that permit operation of housed sensors. For example, a housed optical sensor may transmit light to and from portions of the ear canal through such openings 304. For example, in the depicted embodiment, this housing may include openings at one or more of posterior, front, inferior, anterior and superior locations. In other embodiments, the housing may include a fewer or greater number of openings, which may be located at different locations.

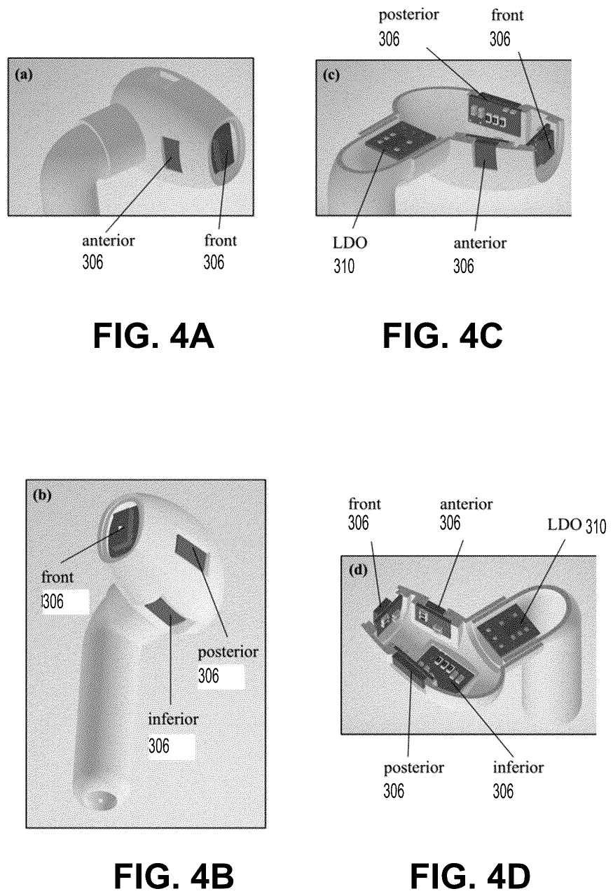

As best seen in FIG. 4A, FIG. 4B, FIG. 4C, and FIG. 4D, one or more of openings 304 may expose an optical sensor 306 for sensing deformation of a corresponding region of the ear canal caused by a lower jaw motion. In the depicted embodiment, optical sensors 306 are disposed at posterior, front, inferior, and anterior openings 304. In some embodiments, optical sensors 306 may be disposed at fewer locations or at additional locations (e.g., the superior location).

In some embodiments, optical sensor 306 is an infrared proximity sensor. As emitted light interacts with the walls of the ear canal, a portion is reflected and detected by the biosensor's photodiode. The quantity of reflected light varies depending on the proximity of the ear canal walls. In this way, volumetric changes in the external auditory canal and movements of the temporomandibular joint (TMJ) may be determined.

In some specific embodiments, optical sensor 306 is a VCNL4020 infrared proximity sensors distributed by Vishay Intertechnology (USA). Upon activation, the sensor emits infrared light into the ear canal, with the emitter drive current adjustable within a range of 10 mA to 200 mA. In the depicted embodiment, an optimal current of 100 mA was selected, taking into account both signal strength and power consumption. Higher values of sensor output indicate proximity to objects and lower values indicate distance. The compact footprint (4.9 mm×2.4 mm) of this sensor makes it suitable for the PCB design in small devices. The 16-bit resolution allows for tracking of fine movements, such as the displacement of 1/10 of a millimeter.

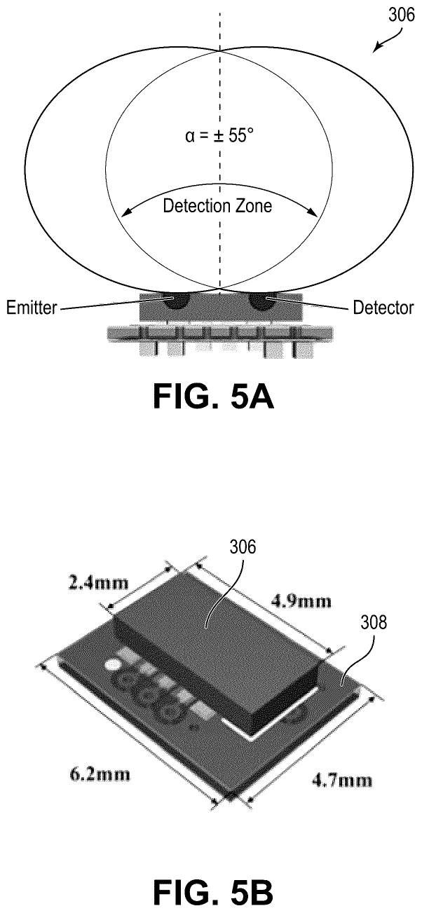

As depicted in FIG. 5A, each optical sensor 306 has a defined sensor coverage. In the case of a VCNL4020 infrared proximity sensor, the sensor coverage is approximately ±55 degrees (as documented by the manufacturer). Namely, the angle of half intensity of the emitter and the angle of half sensitivity of the photodiode are ±55°. Given this coverage per sensor, in the depicted embodiment, sensor assembly 300 includes multiple sensors oriented in defined directions to provide aggregated coverage of deformable segment of the outer ear canal.

In the depicted embodiment, the orientation of a first optical sensor 306 is directed towards the anterior wall. A second optical sensor 306 is positioned to align with the inferior cartilaginous wall of the outer ear canal. Given the 110-degree detection range provided by the first and second optical sensors 306, a third optical sensor 306 is oriented towards the eardrum to encompass the deeper segments of the anterior and inferior walls. A fourth optical sensor 306 is directed towards the posterior wall to serve as a reference point and validate the expectation of minimal or no deformation in the fibrous wall. Referring again to FIG. 3A and FIG. 3B, openings are positioned in the frontal, anterior, posterior, and inferior directions to expose the four optical sensors 306.

In some embodiments, sensor assembly 300 includes a fifth optical sensor 306 at the location of the superior opening 304.

In some embodiments, sensor assembly 300 includes a microphone at the location of the superior opening 304. Such a microphone may be optionally provided to aid data labeling, as it can detect onset and termination of audible activity (e.g., vocal activity).

As shown in FIG. 5B, each optical sensor 306 is mounted on a sensor printed circuit board (PCB) 308. Sensor PCB 308 provides signal and power interconnections for optical sensor 306, which, in some example embodiments, could be a 4.9 mm×2.4 mm VCNL4020 infrared proximity sensor. Other sensors of comparable dimensions and functionality are also applicable to the proposed sensor assembly 300.

As depicted in the cut-away views of FIG. 4C and FIG. 4D, handle assembly 302 includes a power PCB with a low-dropout voltage regulator (LDO) 310, which provides power to in-ear device 102 including to each PCB 308. An LDO provides a tiny, inductor-free power solution that fits on the dedicated power PCB inside the earpiece handle. Fewer passives, no magnetics, and simple routing reduce stack h eight and ease mechanical integration in the ear canal enclosure.

An LDO provides low output ripple and noise, which minimizes baseline wander and noise on the sensor supply that would otherwise modulate the photodiode/readout, improves stability of the downstream 16-bit proximity readings used to detect sub-millimeter motion, and preserves low-frequency fidelity after filtering in post-processing.

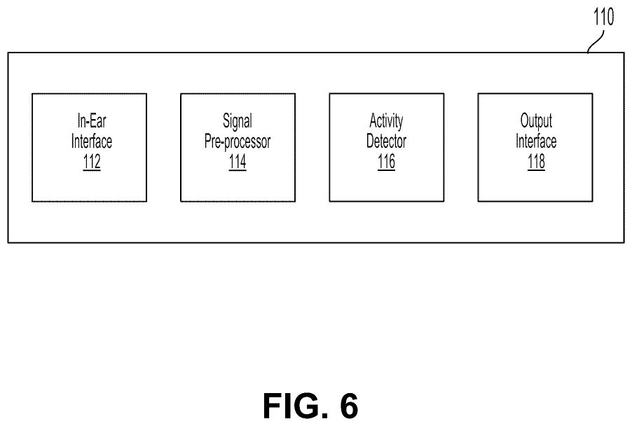

FIG. 6 is a schematic diagram of processing device 110, in accordance with an embodiment. As depicted, processing device 110 includes an in-ear interface 112, a signal pre-processor 114, an activity detector 116, and an output interface 118.

In-ear interface 112 provides a data and/or power link 104 between processing device 110 and each in-ear device 102. For example, in-ear interface 112 provides a link 104L (FIG. 1) between processing device 110 and in-ear device 102L, and a link 104R between processing device 110 and in-ear device 102R. Each of link 104L and 104R may be referred to as a link 104. Link 104 may be a wired link, a wireless link, or a combination thereof.

In embodiments where link 104 includes a wired link, link 104 may be formed of a plurality of conductive wires (e.g., copper wires). Link 104 may, for example, transmit power from processing device 110 to in-ear device 102, e.g., to power its operation and/or charge its battery. In an example embodiment employing a wired link 104, a flexible cable routes from the in-ear device 102 to a small inline dongle containing a processing device 110 in the form of a low-power microcontroller. The dongle aggregates I2C sensor data, timestamps and frames the stream, and presents as a USB device to a host (PC, tablet, phone, or dedicated processor). A USB-C connector supports both data and power delivery. A direct physical connection would provide low complexity in the in-ear device 102, robust power supply, and minimal latency for data transmission.

In embodiments where link 104 includes a wireless link, such link may be formed as a Bluetooth, Bluetooth Low Energy, or similar link. In any event, link 104 provides for data communication between processing device 110 to one or more in-ear devices 102. Such data communication may include, for example, data defining sensor signals sensed at sensors of in-ear device 102 and related handshaking. The sensor signals include, for example, the signals received from one or more optical sensors 106 that sense deformation of a corresponding region of the ear canal caused by a lower jaw motion. Such signals may be referred to herein as deformation signals.

In an example embodiment, a radio system-on-chip (SoC) integrated within the earpiece or within a compact BTE pod packetizes time-stamped sensor signals and transmits them to a processing device using BLE.

In embodiments supporting bilateral deployments, each in-ear device 102 includes a BLE SoC, while one in-ear device 102 operates as a master relay that synchronizes clocks and forwards both left and right sensor signal streams to the processing device 110. Sequence numbers and connection event anchors may be used to maintain inter-ear alignment of the deformation signals.

In-ear interface 112 establishes a communication interface between sensors (e.g., optical sensors 106) of in-ear device 102 and a processor (e.g., a microcontroller) of a processing device 110. In some embodiments, this communication interface may utilize an I2C communication protocol. In some embodiments, the communication interface enables real-time (including near real-time) data communication. In some embodiments, in-ear interface 112 provides a maximum signal sampling frequency of the deformation signals between 20-100 Hz. As will be appreciated, the desired signal sampling frequency depends on the types of user activity to be detected. Specifically, a desired signal sampling frequency may be selected based on expected frequency ranges of signals associated with such user activity and in observation of Nyquist-Shannon sampling theory. In the depicted embodiment, the maximum sampling frequency is approximately 62 Hz, which provides sufficient temporal resolution for speech onset/termination detection while keeping data rate and power at manageable levels for the low-dropout voltage regulator (LDO) 310 and link 104.

In some embodiments, in-ear interface 112 provides a communication interface with a plurality of optical sensors 106. For example, in embodiments where each in-ear device 102 includes four optical sensors 106, in-ear interface 112 integrates data from eight optical sensors 106 (four from in-ear device 102R and four from in-ear device 102L).

In some embodiments, in-ear interface 112 includes an I2C expander (e.g., an Adafruit TCA9548A 1-to-8 I2C Multiplexer), capable of reading data from up to eight connected optical sensors 106.

In some embodiments, in-ear interface 112 includes an optional comparator circuit and connectors for a microphone that can be used to detect the presence of the audible speech and aid data labeling. This comparator circuit compares signals from the microphone to a set reference voltage (which can be adjusted by potentiometer) and outputs high-voltage level if speech is present and low-voltage level if there is no speech.

Signal pre-processor 114 is configured to receive sensor signal data from in-ear interface 112 and apply pre-processing. For example, signal pre-processor 114 may apply signal filtering, e.g., to within the range of 0.5 Hz to 10 Hz which corresponds to the lower jaw movement. In some embodiments, signal pre-processor 114 may apply another suitable form of signal conditioning. In some embodiments, signal pre-processor 114 may apply normalization to bring signal data into a normalized range (e.g., [0, 1]).

Activity detector 116 processes sensor signal data to generate an output signal corresponding to the lower jaw motion.

In some embodiments, the output signal may encode a particular user intent. The particular user intent may include at least one of a speech intent, a chewing intent, or a swallowing intent.

In some embodiments, the output signal encodes a particular user activity. The particular user activity may include at least one of a particular unit of speech, a particular unit of chewing, a particular unit of smiling, a particular unit of laughing, a particular unit of yawning, or a particular unit of swallowing. The particular user activity includes a particular kissing.

In some embodiments, the output signal encodes a particular user request or command.

Output interface 118 is configured to provide the generated output signal to another device, e.g., by a wired or wireless link. For example, the output signal may be used as a control signal for an artificial speech generator based on particular units of speech that are detected.

In some embodiments, activity detector 116 generates the output signal by applying a machine learning model to the deformation signals in inference mode. In some embodiments, the machine learning model includes a neural network trained to map deformation signals to output signals.

In some embodiments, the machine learning models are trained on labeled proximity data acquired from optical sensors embedded in the outer ear canal to distinguish speech from other activities such as chewing, yawning, coughing, smiling, laughing, and background noises. Ground-truth labels are established through two complementary strategies: manual segmentation of proximity signals based on pre-phonatory and post-phonatory activities and sound-aligned segmentation that maps the onset and termination of audible speech acquired through the built-in microphone to the proximity sensor data. This dual labeling captures the full temporal envelope of mandibular behavior, including activation that precedes audible phonation and persists briefly after it ends.

In some embodiment, activity detector 116 is configured to differentiate speech activity from other non-speech activities, such as mastication or coughing. Such differentiation may be used to activate an output interface 118, e.g., for transmitting a detected unit of speech to a sound transducer (e.g., electrolarynx) to generate the artificial voice.

Each of in-ear interface 112, signal pre-processor 114, activity detector 116, and output interface 118 may be implemented using a suitable combination of software and hardware components.

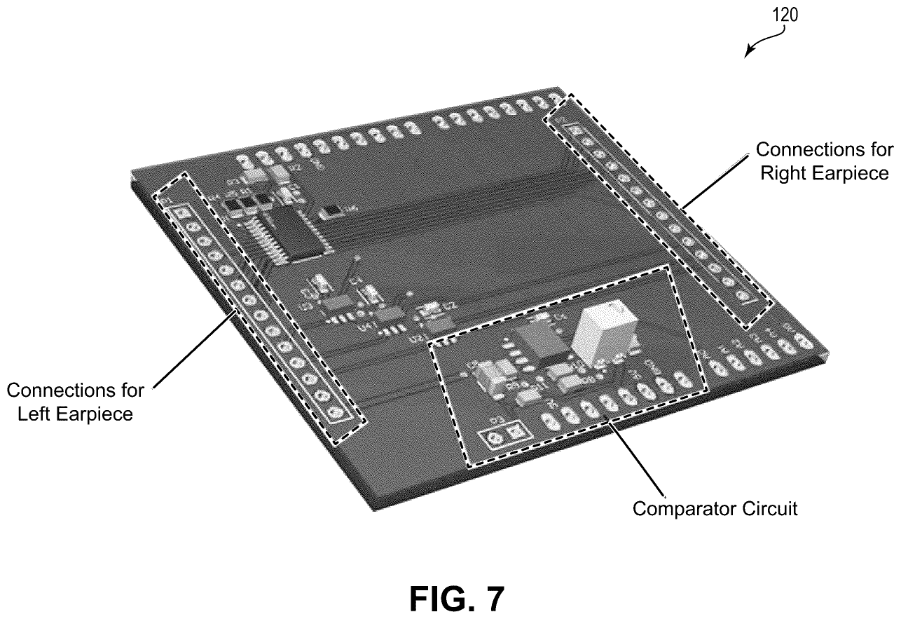

In some embodiments, such hardware components may include a master PCB 120 with a connection port for establishing a link to in-ear device 102L and a connection port for establishing a link to in-ear device 102L, and the noted comparator circuit, as shown in FIG. 7.

In some embodiments, such software components may be implemented in whole or in part using conventional programming languages such as Java, J #, C, C++, C#, Perl, Python, Visual Basic, Ruby, Scala, etc. Such software components of system 100 may be in the form of one or more executable programs, scripts, routines, statically/dynamically linkable libraries, or servlets.

### Experiment #1

### Experimental Procedure

An experiment using an embodiment of monitoring system 100 was carried out in two distinct, time-separated trials, during which an in-ear device 102 was removed from the ear canal and reinserted at the onset of each trial. This approach was implemented to ensure to guarantee the reproducibility and reliability of the data, especially when evaluating performance of monitoring system 100 during dynamic activities such as speaking, coughing, and chewing. The 15-minute interval between trials was crucial to minimize any lingering effects from prior activities and to establish a consistent baseline for each trial. This duration ensured sufficient time to reset the physiological state without introducing unrelated variability. The experimental tasks were grouped in 3 categories: fundamental mandibular movements (FMM), integrated mandibular movements (IMM), and articulatory mandibular movements (AMM).

Fundamental mandibular movements included lower jaw protrusion, retraction, elevation/depression, and lateral movement (sliding left and right). These movements are considered fundamental as they represent building blocks of more complex, integrated activities, such as eating or speaking. The rationale behind studying these movements is to facilitate the identification of the structural elements involved in the execution of more complex integrated mandibular movements. Integrated mandibular movements in this study included mastication of two substances with different consistencies (cracker and gum) and yawning. To test mandibular (articulatory) speech dynamics, subject were tasked with reading material from the well-established, phonetically-balanced, structured speech tests like Harvard Sentences, Assessment of Intelligibility of Dysarthric Speech (AIDS), vocalize sustained vowels (a, e, i, o, u), and read some of the popular, phonetically-balanced phrases used in everyday speech, compiled based on the statistics of usage. The speech material was selected carefully to include prompts at the level of phonemes (vowels), single word building up to 7-words long sentence (AIDS), full phonetically balanced sentence (Harvard Sentences), and a variety of declarative, interrogative, imperative, exclamatory, and conditional phrases. The goal was to determine the sensitivity of monitoring system 100 to the amount of the speech material. In other words, it was desired to determine if monitoring system 100 can detect smaller units of speech, such as phonemes or single words, or only speech activity as a whole. Monitoring system 100 was operated in an acoustically insulated room.

### Measurement Setup

As a reference measurement, a 3-channel sEMG device and audio measurement were used. TMSi SAGA (TMSi International, Netherlands) sEMG acquisition system and a YETI Multi-pattern USB microphone (Logitech, Switzerland) were used. The bipolar sEMG electrodes were positioned on muscle Masseter and submental space to ascertain the movements of the lower jaw that are typically caused by mastication and coughing, while the 64-contact, high-density flexible sEMG electrode was placed on muscle Mentalis to detect the movements of the mouth related to speech articulation. The table-mounted microphone was used to confirm the propagation of the sound detected at 50 cm to the lip corner and to ensure the sync between all the other modalities. Sampling frequency of the sEMG system was 4096 Hz, while audio was acquired with the sampling frequency of 44.1 KHz and further down-sampled to 24 kHz.

The three modalities (proximity, sEMG, audio) were simultaneously recorded on the same computer functioning as the end-device and were synchronized with the system clock of the PC to guarantee temporal alignment. As an extra measure, all the time stamps produced during the recording were stored alongside the data for each modality. Subsequently, the time stamps were cross-referenced post-experiment to validate the alignment of the data.

Outer Ear Canal Deformation with Respect to Sensor Orientation

To compare the deformation occurring in various sensor orientations, the collected proximity data underwent initial filtering within the range of 0.5 Hz to 6 Hz, which corresponds to the lower jaw movement, followed by normalization [0,1] range.

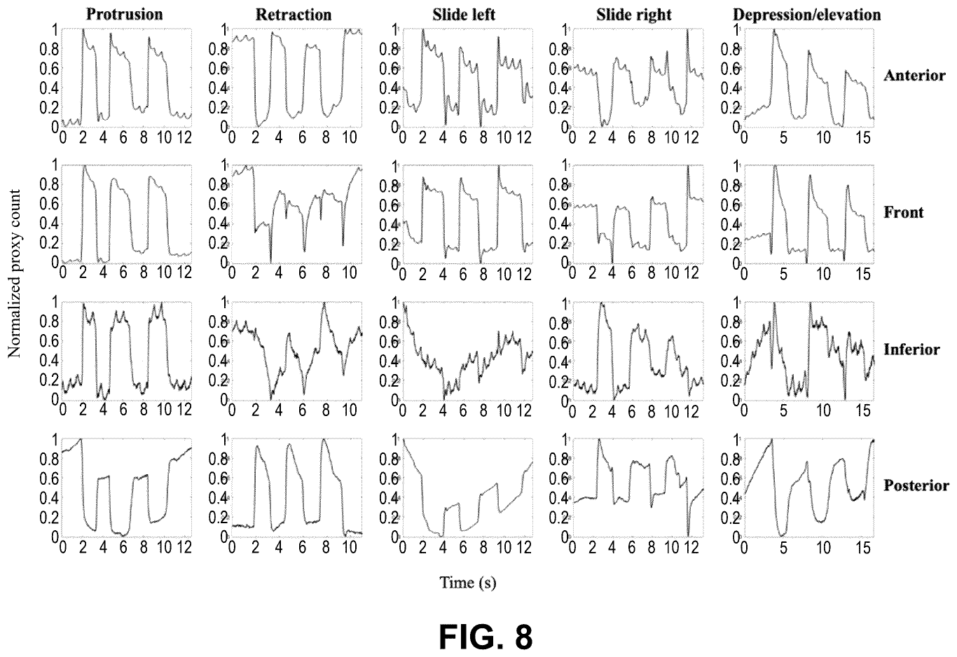

Subsequently, an analysis of fundamental mandibular movements was conducted to discern the fundamental components of lower jaw displacement. The findings of this analysis, illustrating various pairs of activities and sensor positions, are presented in FIG. 8, which shows deformation in the right outer ear canal of a subject performing fundamental mandibular movements. Each movement was repeated three times consecutively. Columns represent movement type, while rows correspond to sensor orientation. Normalized proximity data count [0,1] is shown as a function of time, where this count is proportional to amplitude of the sensed signal.

This approach allowed for a comprehensive examination of the directional variations in sensor readings during lower jaw movements, providing valuable insights into the distinct patterns of deformation captured by the sensors. It is clearly visible that the data from the anterior and frontal sensor orientations provides the most stable reading of the movement, in contrast to the inferior and posterior sensor which exhibit greater level of noise, variable trend, and irregularity in the captured information.

Comparison of the Right and Left Outer Ear Canal with Fundamental Mandibular Movements

As noted above, volumetric change in the two ear canals can be symmetrical or asymmetrical in both amplitude and direction of change. To investigate this, a comparative analysis was performed of event onset and termination times for fundamental movements in the right and left ear canals.

Initially, the absolute normalized first derivative was computed to evaluate abrupt changes in the signal, which are indicative of onset and termination instances. Then the most prominent peaks were identified in the data by applying experimentally derived threshold set at 25% of the maximum peak value. This threshold value resulted in 4 missed peaks out of 24 in the left ear canal and 2 missed peaks out of 24 in the right ear canal. However, this was a justified trade-off, as any further reduction of the threshold value led to a substantial increase in false positive peaks.

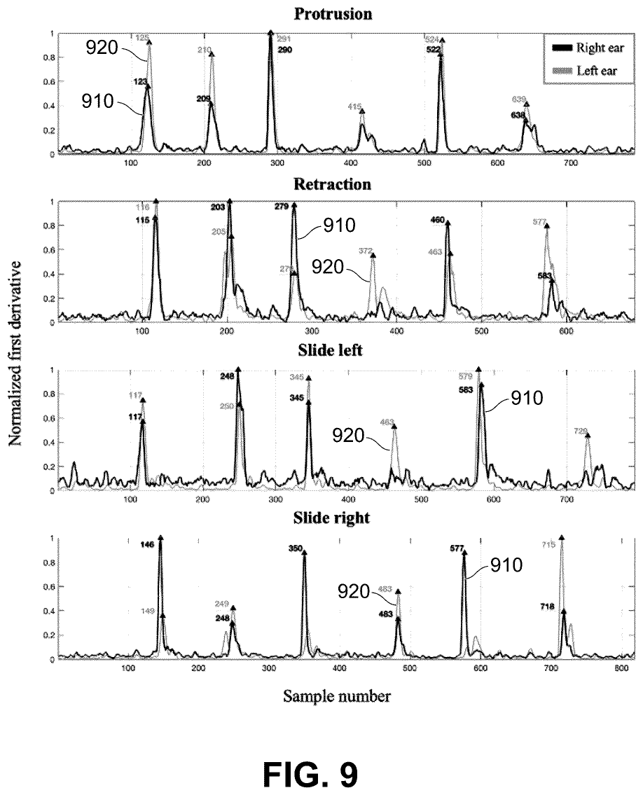

FIG. 9 shows a cross comparison of the magnitude of change in the right (910) and left (920) ear canal, with fundamental mandibular movements, with the front sensor orientation. Horizontal axis represents a sample number, while the vertical axis represents the absolute normalized first derivative of the proximity signal. The threshold used to detect peaks in the first derivative was 25% of the maximum peak value. Maximum observed lag between the two signals, or in other words, between the left and right ear canal is five samples or 80 ms. The cross-comparison shown in FIG. 9 allows comparison of the amplitude of change as well as the maximum lag between the two ear canals for the front sensor.

### Integrated Mandibular Movements

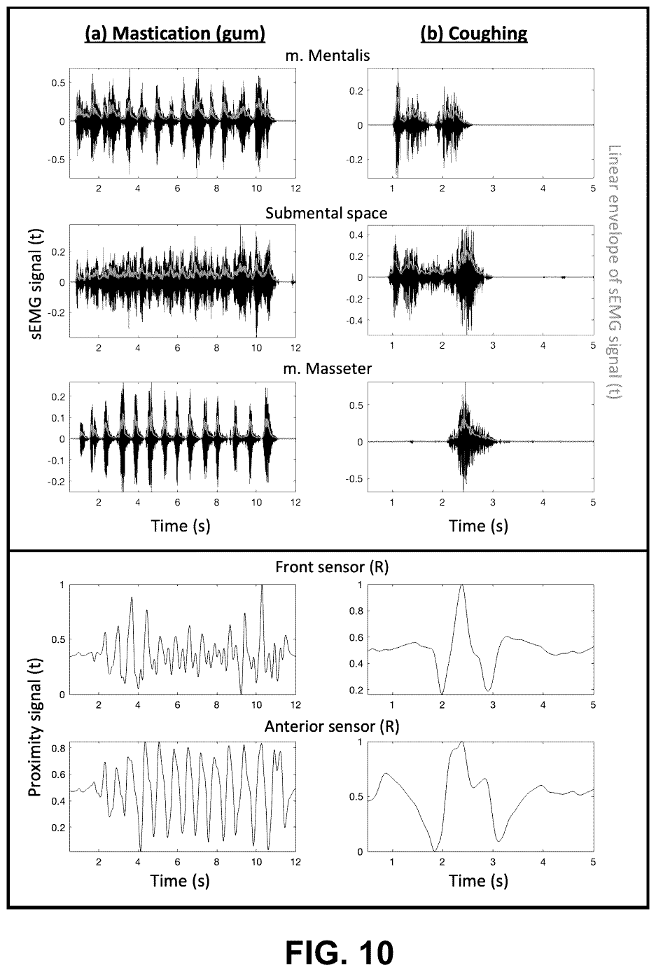

The integrated mandibular movements captured in the outer ear canal using the proposed proximity-based system were compared to the well-established sEMG measurement. FIG. 10 shows a 12-second long mastication segment (left panel) and a 5-second long coughing segment (right panel). Proximity measurement (bottom box) from the right ear canal (R) and two different sensor orientations (front and anterior) was compared with sEMG recordings from muscle Mentalis, Submental space, and muscle Masseter (top box). The red line indicates the linear envelope of the filtered sEMG signals. sEMG signals were sampled with a frequency of 4096 Hz, while proximity signals were sampled at 62 Hz.

Through the comparison of linear envelopes derived from the sEMG measurements over the Mentalis muscle, Submental space, and Masseter muscle with simultaneously recorded proximity data, it was desired to evaluate the capability of the proposed device to capture segments of compound movements, such as mastication or coughing, and to identify any potential delays between the two modalities. The results depicted in FIG. 10 demonstrate that the proximity sensors were able to capture segments of compound movements.

Specifically, both the front and anterior proximity sensors detected chewing with a slight delay of approximately 0.5 seconds. In the case of coughing, the proximity reading closely resembled the sEMG reading from the masseter muscle, and even preceded the sEMG onset. Notably, in both mastication and coughing, the anterior sensor provided a clearer and less noisy reading.

### Mandibular Speech Dynamics

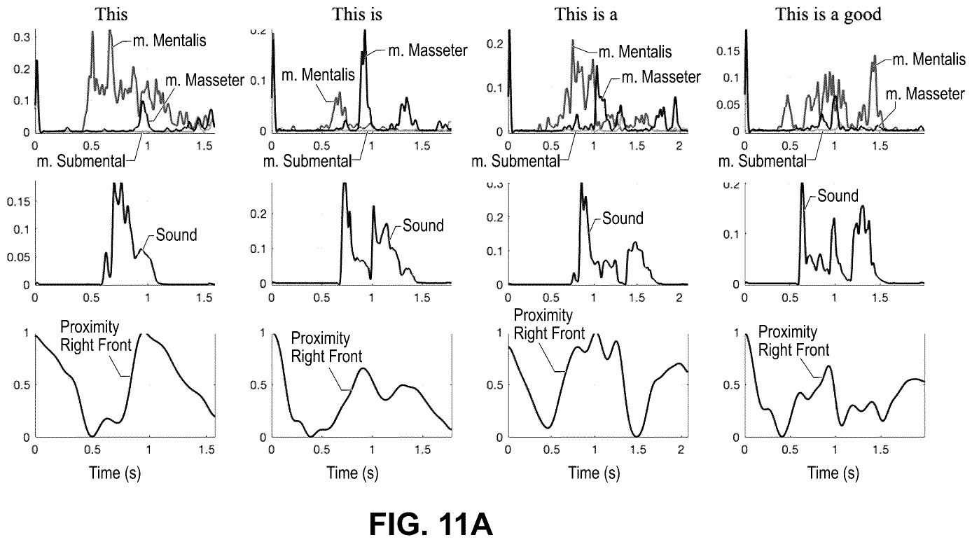

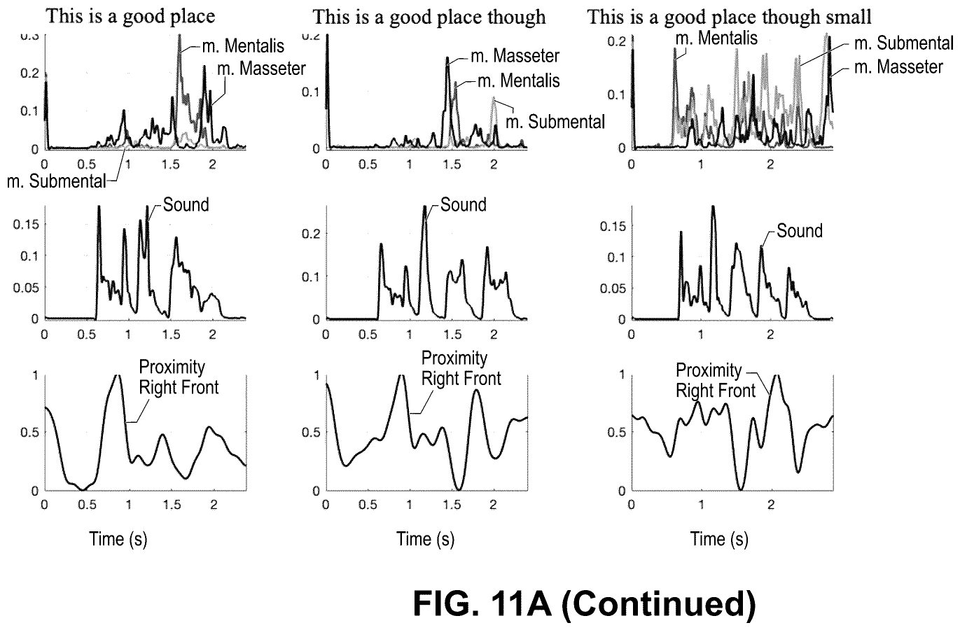

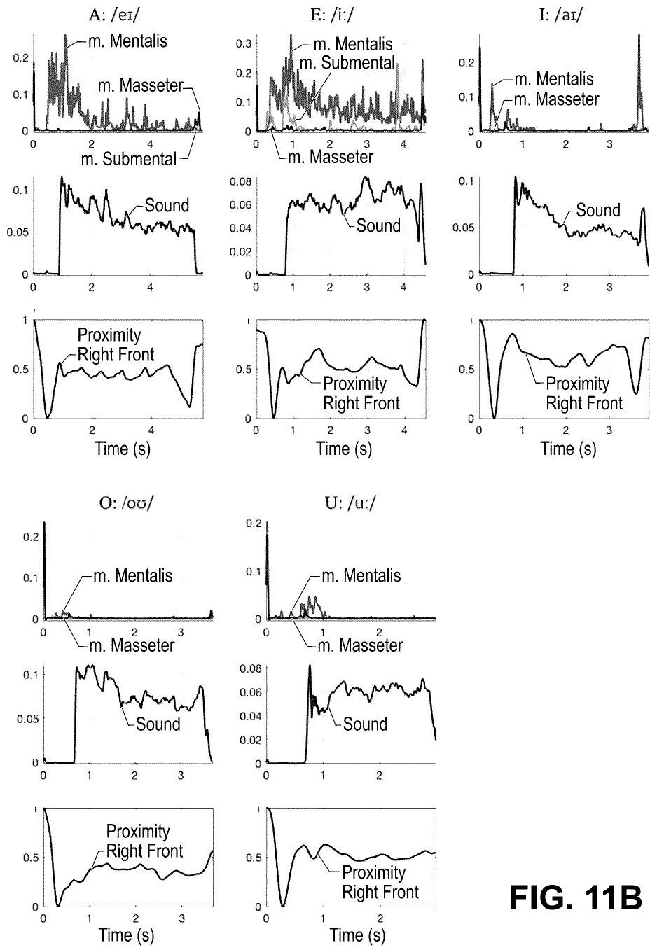

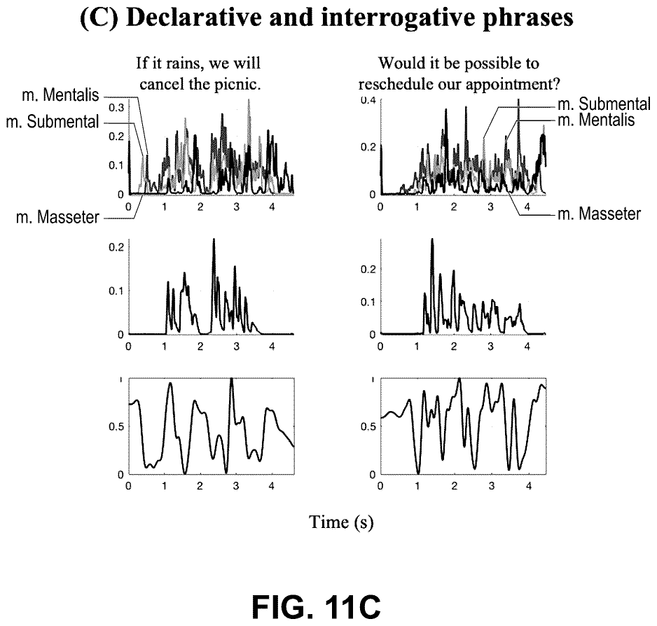

Mandibular speech dynamics were assessed through a series of structured, standardized speech tests. Given the stochastic and highly nonlinear nature of speech, as well as its lack of periodicity (unlike in mastication), it was desired to determine whether the deformation in the outer ear canal would remain present and detectable during speech production. Additionally, it was desired to investigate whether there exists a word or phoneme threshold (see FIG. 11A) that triggers a minimum detectable volumetric change in the ear canal during speech. The recorded proximity data was cross-referenced with muscle and sound envelopes.

Proximity signal was recorded in the right ear canal, with the front orientation of the sensor cross compared with linear envelopes extracted from sEMG and sound recordings.

The results of AIDS test are shown in FIG. 11A, demonstrating a buildup of a 7-word sentence, while vocalization during sustained production of 5 vowels is shown in FIG. 11B. An example demonstrating the detection of declarative and interrogative phrases is presented in FIG. 11C. The findings indicate that the proximity sensors, particularly the front sensor in this instance, can capture the instances of speech onset and termination at the level of vowels, single words, and full sentences, which align with the sound and sEMG signals at onset/termination times.

Cross Comparison with the Sound and Reproducibility

Proximity data were compared to sound measurements to assess the time shift between the two modalities.

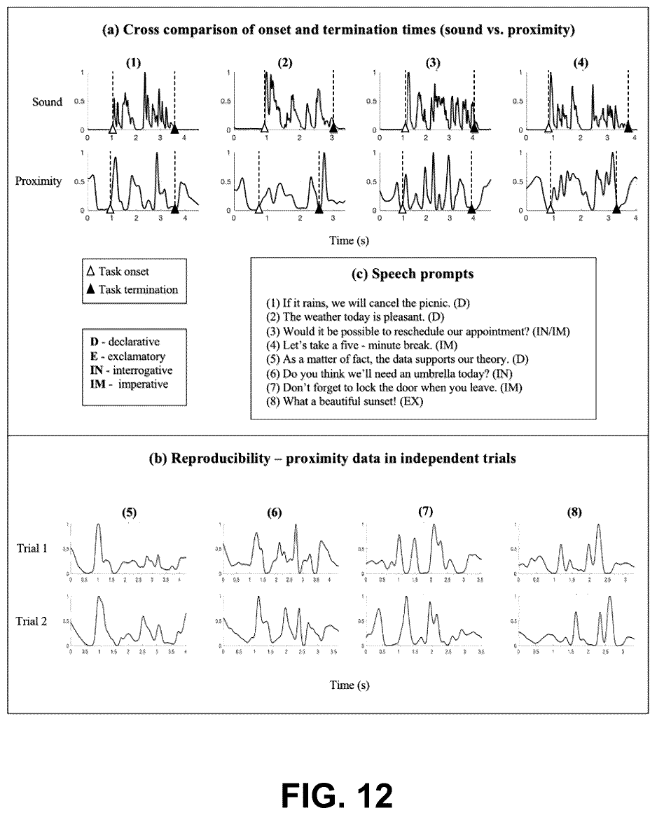

FIG. 12 depicts assessment of the proximity data from front sensor in the right ear. FIG. 12—Panel A shows Comparison of onset and termination instances detected in proximity data with those of sound envelope. The instances were detected by using dynamic threshold based on the short-term signal energy. FIG. 12—Panel B shows proximity data collected in two independent trials. The device was taken out of the ear canal in-between trials to eliminate potential bias. FIG. 12—Panel C shows speech material used for the analysis of onset/termination times and reproducibility. Prompts include variety of declarative, exclamatory, interrogative, and imperative sentences.

As illustrated in FIG. 12—Panel A, the onset and termination times detected from the volumetric change in the right outer ear canal closely align with those of the sound envelope. The onset and termination times were identified using a dynamic threshold based on the short-term energy of the signal windows. For sound measurement, signal was segmented in 10 ms long frames, while for proximity measurement 50 ms long frames were used.

Furthermore, the reproducibility of monitoring system 100 was assessed by recording a test subject uttering the same phrases in two independent trials. In the second trial, the device was removed from the ear and repositioned to mitigate any potential bias stemming from the specific position of the earpiece inside the ear canal. FIG. 12—Panel B demonstrates how the device can replicate signals for prompts corresponding to declarative, interrogative, imperative, and exclamatory sentences (FIG. 12—Panel C).

Upon comparing the onset and termination times in the proximity and sound recordings, it becomes evident that in nearly all cases, the detected proximity onset from inside the outer ear canal preceded the audible sound. Depending on the characteristics of the speech material (voiced/unvoiced segments), proximity termination instances could occur before the termination instance of the audible sound.

Visual examination of several speech prompts in independent trials reveals characteristic peaks for each prompt, which could be reproduced in the independent trials.

### Sensor Orientation

The findings align with existing literature regarding the deformation of the external auditory canal due to mandibular movements. Specifically, the data obtained from the anterior and frontal sensor orientations consistently demonstrate the most significant changes. In contrast, the inferior and posterior sensor orientations also capture movement information, although with greater variability and irregularity. Nevertheless, retaining all four sensors may prove valuable for future measurements, as it presents the potential for sensor fusion and the comprehensive reconstruction of volumetric changes in the lower jaw.

### Comparison of the Two Ear Canals

Data gathered from both the left and right ear canals validate existing literature, which states the presence of variations in both the magnitude and direction of volumetric change within the two ear canals. The maximum observed delay between the two signals was 80 milliseconds during fundamental mandibular movements.

### Fundamental Mandibular Movements

The movements of protrusion, retraction, depression/elevation, and lateral movement have each exhibited distinct characteristics in both ear canals. Furthermore, the direction of these changes varies between the two ears across different tasks. For instance, when considering the sensor with an anterior orientation during retraction, both the left and right ear canals demonstrated a decrease in proximity count, indicating an increase in the distance from the sensor to the anterior wall of the ear canal. Conversely, during protrusion, a positive change in the signals was observed, suggesting a decrease in distance. Additionally, a positive change direction was observed in the right ear canal during sliding left, while the left ear canal exhibited a negative change. Conversely, during rightward sliding, the opposite pattern was observed. Understanding the fundamental movements of the mandible is significant as these movements serve as building components of more complex tasks such as mastication and speech.

### Integrated Mandibular Movements

In comparison to sEMG, proximity signals exhibited the closest resemblance in terms of activation and termination times to those of the masseter muscle. Nonetheless, a slight delay between sEMG and proximity signal recordings was evident, which is consistent with the distinction between electrical and mechanical activity. Furthermore, the temporal lag between muscle activation and the transmission of movement to the temporomandibular joint was also a contributing factor to this disparity.

### Mandibular Speech Dynamics

The findings from the analysis of mandibular speech dynamics indicate that speech signals were discernible even at the level of individual words. Specifically, a single word elicited a discernible deformation in the outer ear canal, which the prototype device was capable of detecting. The effectiveness of detection, particularly in terms of onset and termination times, may have depended on the phonetic characteristics of the speech material, specifically whether it was voiced or unvoiced. It is worth noting that the proximity onset precedes the audible sound, which enables monitoring system 100 to provide a hands-free control signal for artificial voice systems following a laryngectomy.

In the context of vowel testing, the device was able to detect each of the five tested vowels. Moreover, sustained vowels such as “I,” “O,” and “U” exhibit discernible patterns in the proximity data that are not evident in the surface electromyography data.

Moreover, the reproducibility of the proximity data recorded by the prototype device is evident across independent trials, during which in-ear device 102 was removed from and reinserted into the ear canal. This observation was consistent across a variety of declarative, exclamatory, interrogative, and imperative sentences.

### Experiment 2

An embodiment of monitoring system 100 underwent evaluation involving a larger cohort of participants to assess its effectiveness in identifying speech intentions and generating control signals for artificial speech systems. Analytical methods were applied to examine the properties of the proximity signal, and machine learning models were developed to distinguish speech from other orofacial activities, which represent potential sources of false triggers for the device. Additionally, findings on the (a) symmetry between the two ear canals observed in all subjects are presented.

### Subject Cohort

Demographic information pertaining to the recruited participants is presented in Table 1.

TABLE 1
Subject cohort (N = 10).

| Subject | Gender | Age | Ethnicity |
|---|---|---|---|
| S1 | F | 27 | Caucasian (European) |
| S2 | F | 29 | Asian (Indian) |
| S3 | F | 28 | Caucasian (European) |
| S4 | M | 28 | Caucasian (American) |
| S5 | F | 27 | Asian (Indian) |
| S6 | F | 27 | Caucasian (Iranian) |
| S7 | M | 22 | Asian (Thai) |
| S8 | F | 31 | Caucasian (European) |
| S9 | M | 22 | Asian (Chinese) |
| S10 | M | 26 | Hispanic/Latino |
| | | | (Mexican) |

### Experiment

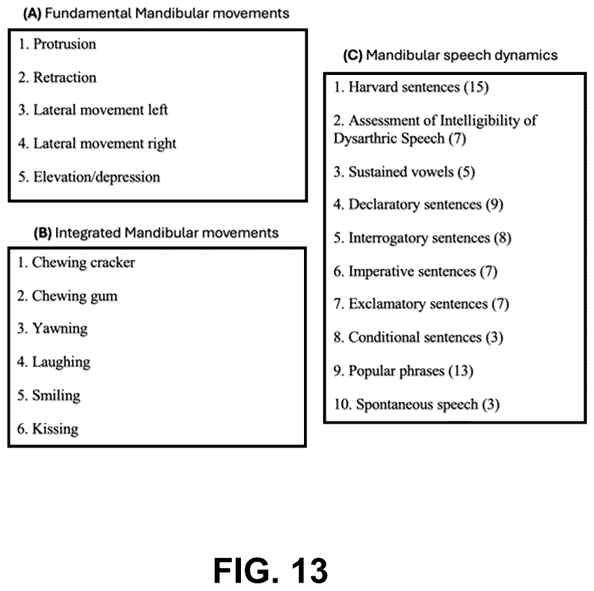

The research methodology adhered to a structure similar to the framework delineated in Experiment 1. The tasks were once again categorized into three distinct groups: FMM, IMM, and AMM. While the fundamental mandibular movements (FIG. 13—Panel A) remained consistent with Experiment 1, the list of IMM was broadened by the inclusion of additional non-speech activities involving the lower jaw (FIG. 13—Panel B). Each task within the FMM and IMM group was executed five times, with brief (few seconds) intervals between repetitions.

The assessment of mandibular speech dynamics was performed by using the identical speech material as detailed in FIG. 13—Panel C. Furthermore, three open-ended questions were incorporated, prompting subjects to respond in their natural conversational style, to assess the device's efficacy in capturing spontaneous, unscripted speech. In addition to vocalizing the speech prompts, participants were instructed to articulate the same content (excluding spontaneous speech) silently. This segment of the protocol aimed to simulate the conditions of laryngectomy, where individuals have intact articulatory function but lack vocalization, and will be referred to as SAMM (silent articulatory mandibular movements).

The entire protocol was repeated twice, resulting in two trials per subject. A 15-minute intermission separated the two trials, during which the device was removed from the ear canal and repositioned for the subsequent trial. This procedure was implemented to ensure the reproducibility of device placement and to mitigate any potential influence of device shifting during the initial trial on the data integrity. The intermission also provided a standardized rest period, minimizing any variability in participant fatigue or device performance across trials.

FIG. 13 depicts experimental tasks used in the experiment with proximity data: Panel A shows fundamental mandibular movements, encompassing protrusion and retraction, which are movements of the lower jaw forward and backward, respectively; Panel B shows integrated mandibular movements, which include six non-speech orofacial activities; Panel C shows mandibular speech dynamics featuring phonetically balanced materials such as Harvard Sentences and the Assessment of Intelligibility of Dysarthric Speech (AIDS), sustained vowels, and a range of commonly used declarative, interrogative, imperative, exclamatory, and conditional phrases. Spontaneous speech is represented by responses to three questions asked by the experimenter. The numbers next to each prompt group indicate the quantity of sentences in that group.

### Hardware

The study simultaneously captured data from three modalities: proximity, surface electromyography (sEMG), and audio. The sEMG signals were acquired using the TMSi SAGA sEMG acquisition system by TMSi International (Netherlands), while audio recordings were obtained through the YETI Multi-pattern USB microphone manufactured by Logitech (Switzerland).

The sEMG system comprised 4 bipolar electrodes strategically placed over the zygomaticus major, masseter, submental space, and neck strap muscles, in addition to a high-density sEMG electrode situated on the chin area. The microphone was positioned on the table in front of the participant alongside a screen displaying speech prompts. The sampling frequencies for the different modalities were set at 62 Hz for the proximity unit, 2,000 Hz for sEMG, and 44.1 KHz for audio recordings.

While proximity served as the primary modality under investigation, the sEMG and audio data were utilized as reference points. A personal computer facilitated the recording, visualization, and storage of data from all three modalities, ensuring synchronization with the system clock. To enhance data integrity, timestamps generated during recording were stored alongside the data for each modality. Post-experiment, these timestamps were cross-referenced to validate data alignment.

### Dataset Structure and Data Preprocessing

Upon collection, the data was structured to encompass proximity, surface electromyography (sEMG), and audio data for each participant across the four segments of the protocol. The number of recordings per trial per individual modality varied, with 5, 6, 77, and 74 recordings for FMM, IMM, AMM, and SAMM, respectively. The sEMG dataset comprised a total of 68 channels (64 corresponding to the high-density electrode and 4 bipolar channels), resulting in 22,168 recordings over the course of two trials. In contrast, the proximity unit featured 8 channels (4 allocated to each ear canal), yielding a total of 2,608 recordings across the two trials. The audio component contributed 326 recordings in total.

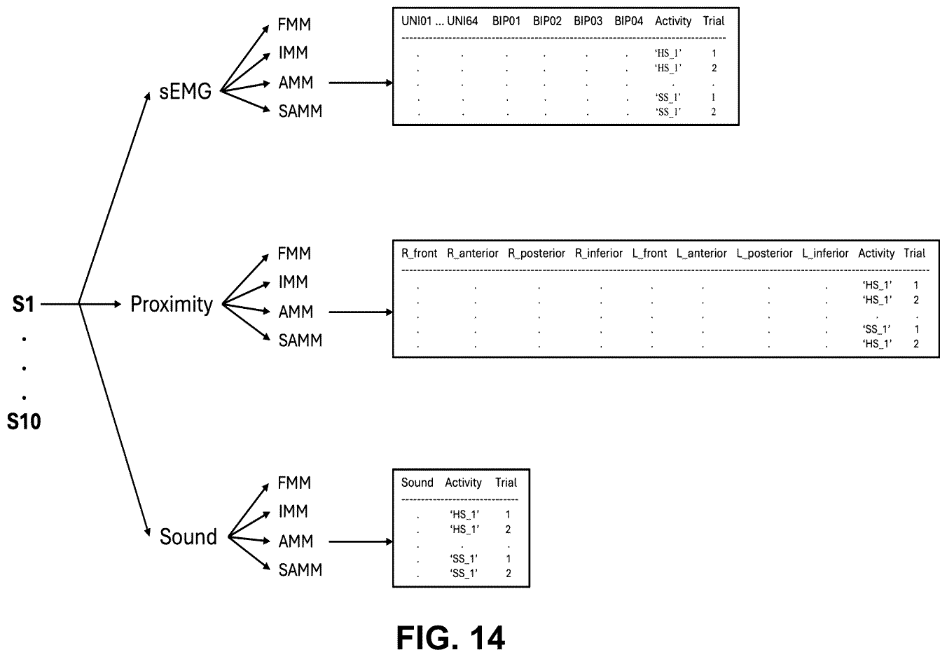

FIG. 14 depicts the dataset structure. The dataset contained a total of 25,102 recordings, out of which 22, 168 are contributed by sEMG, 2,608 by proximity, and 326 by sound. In the figure FMM is the fundamental mandibular movements, IMM is the integrated mandibular movements, AMM is articulatory mandibular movements, and SAMM is the silent articulatory mandibular movements, S1 to S10 denote data from 10 different subjects, while HS_1 provides an example of labels for a specific activity, in this case Harvard Sentence number 1.

The data processing approach involved three distinct streams to handle the unique characteristics of each modality before combining them. The preprocessing of the sEMG data involved the identification and elimination of artifacts in both the time and frequency domains through the implementation of a sliding window technique.

The audio data underwent downsampling to a frequency of 24 kHz without further processing at this stage. Preprocessing the proximity data posed a non-trivial challenge due to its inherently low-frequency characteristics. The raw signals were examined in both the time and frequency domains. This analysis included waveform assessment, periodicity evaluation, power spectrum analysis of baseline signal and selected activities.

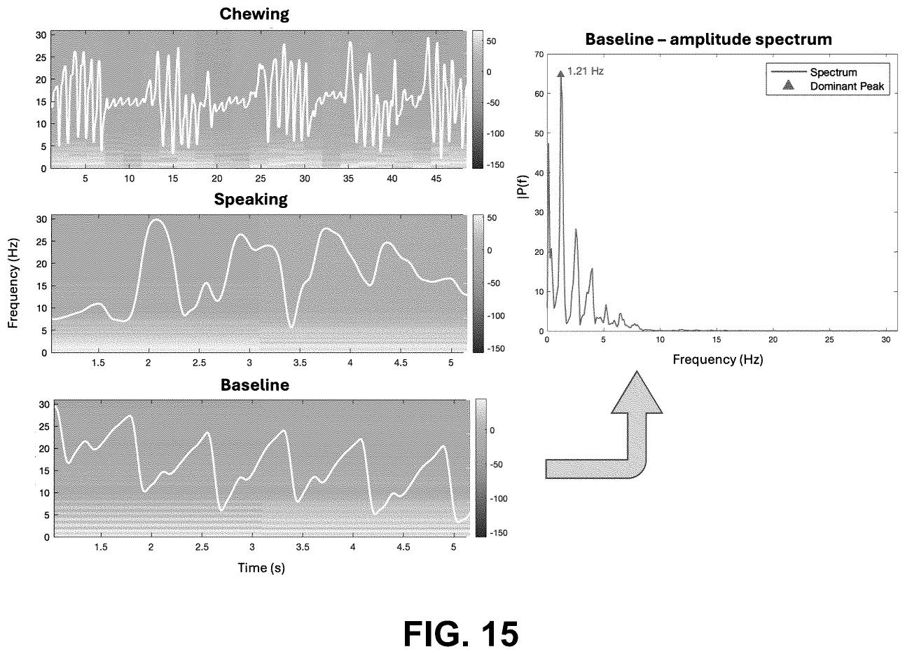

The dominant signal detected during the baseline phase in both ear canals was identified as presumably heartbeat, which was further corroborated through spectral analysis. During active sequences in the data, it was observed that the frequency spectrum extended up to approximately 10 Hz (FIG. 15), prompting the selection of this frequency as the low-pass cut-off for a 4th order Butterworth filter applied to the data. Initially, a high-pass cut-off frequency of 0.5 Hz was trialed based on existing literature recommendations. However, this setting resulted in the exclusion of low-frequency peaks occurring at task initiation and cessation points. Subsequently, the high-pass cut-off frequency was adjusted to 0.1 Hz to encompass the critical low-frequency peaks (0.1-0.5 Hz) while effectively eliminating zero offset and trends in the data. Filtered proximity data was smoothed using a moving average filter with window size of 6 samples.

FIG. 15—right panel depicts a spectrogram of proximity data during chewing and speaking, showing that most of the proximity energy is in the low-frequency range (<10 Hz). FIG. 15—left panel depicts amplitude spectrum of the baseline signal recorded in the ear canal, showing that the dominant signal in the absence of the activity is heartbeat. Accordingly, in some embodiments, monitoring system 100 may be configured to be used for heart rate monitoring.

### Data Segmentation

The first step of data segmentation was the identification of active sequences within the dataset. To enhance the Signal-to-Noise Ratio (SNR) and refine edge detection (onset/termination), the sEMG and audio data underwent preprocessing utilizing the Wiener-Scalart method. Subsequently, an envelope-threshold methodology was implemented. This approach demonstrated notable efficacy in detecting active sequences within sEMG and audio data, which was further validated by its capacity to generalize and perform effectively on new data acquired using a different data acquisition system. Identifying active sequences within the proximity data was not a trivial task.

The following two strategies were used.

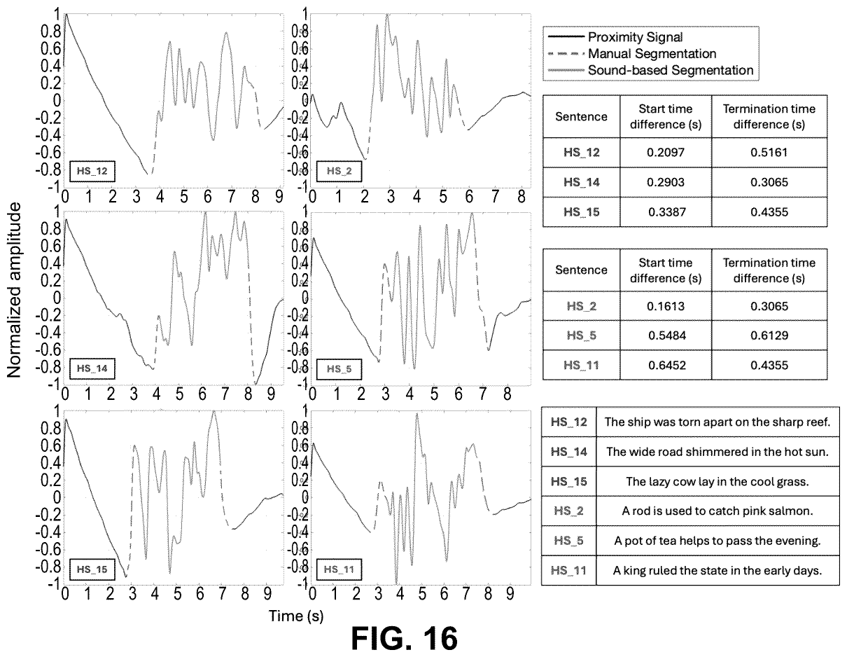

Manual segmentation—marking of onset/termination times for all experimental segments. Fundamental and integrated mandibular movements displayed distinct onset and termination points, facilitating straightforward segmentation. Mandibular speech movements exhibited a slightly different behavior (as illustrated in FIG. 16). Across various subjects and speech tokens, it was consistently observed that the proximity signal exhibits a downward trend prior to speech onset and an upward trend following speech termination. Consequently, signal onset and termination were systematically identified and marked based on the local minimum of these trends.

For articulatory mandibular movements, i.e., speech, the study leveraged known onset/termination times of audible sound and scaled them to the frequency of proximity data to label the corresponding active segments in the data. The rationale behind this approach lies in the necessity for the prospective control signal to activate and remain active throughout the duration of the audible sound. Thus, the points in the proximity data that align with the times of sound activation are expected to be marked as active.

FIG. 16 depicts a comparison between manual segmentation and sound-based segmentation of proximity data. Manual segmentation relies on visible activity in the proximity signals, while sound-based segmentation is derived from audible speech cues. Differences in the onsets and terminations of the proxy and sound data are presented on the right for Harvard Sentences (HS) starting with ‘The’ (HS_12, HS_14, HS_15) and ‘A’ (HS_2, HS_5, HS_11).

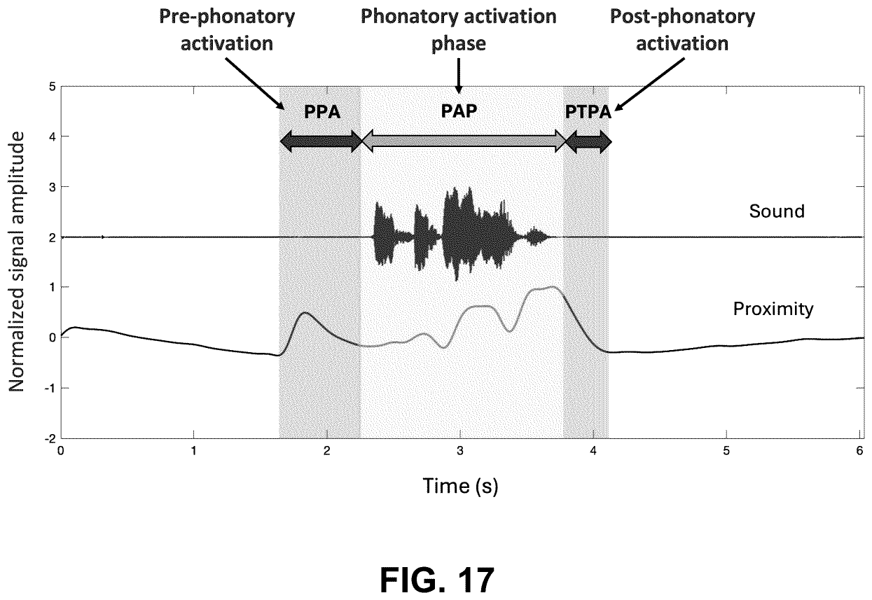

FIG. 17 depicts normalized sound and proximity signals illustrating the pre-phonatory and post-phonatory activation (PPPA) in the proximity data, which precedes and extends beyond the audible sound duration. Additionally, the phonatory activation phase (PAP) shows the proximity activation coinciding with sound production.

As shown in FIG. 17, the manually annotated segments within the proximity data exhibit a consistent pattern of commencing prior to the onset of audible sound production and extending beyond the cessation of the audible sound. This observed phenomenon persists across all speech prompts and study participants. The segments observed before and after the audible sound likely pertain to preparatory actions associated with speech articulation, such as the adjustment of articulators, including the mandible, to achieve the requisite position for a specific phoneme. The manually labeled segments will be referred to herein as pre-phonatory and post-phonatory activation (PPPA) in the proximity data, while the segments aligning with active sequences identified through sound activation times will be referred to herein as phonatory activation phase (PAP).

### Feature Extraction

The magnitude of motion group encompassed five features: Root Mean Square (RMS), Variance, Entropy, Peak Power, and Power Spectral Density, with the primary objective of encoding the energy content within data segments. On the other hand, the periodicity of motion group comprised seven features aimed at encoding dominant frequency information within the data segments. These features included Zero Crossing, Variance of Zero Crossing, Number of Auto-correlation Peaks, Prominent Peaks, Weak Peaks, Maximum Autocorrelation Value, and First Peak.

The waveform features of proximity signals were characterized by extracting a set of 12 features referred to as waveform features. This feature group encompassed parameters such as Crest Factor, Form Factor, Waveform Length, offering an understanding of the waveform shape. Parameters like Rise Time and Fall Time capture the duration for the signal to ascend and descend between thresholds, reflecting the speed of signal changes, while Mean Absolute Deviation provides a measure of variability in the signal. Additionally, Signal Slope Sign Changes and Zero Crossing Rate evaluate the signal's oscillatory behavior and frequency content, respectively. The Integral of Absolute Value sums the absolute signal values, giving a sense of overall signal magnitude, and Variability shows amplitude variance. The Number of Peaks counts the signal peaks to infer frequency and regularity, and the Root Mean Square of Successive Differences analyzes the variability of the differences between successive signal values, crucial for understanding the dynamics induced by lower jaw movements in the ear canal.

The spectral analysis of the signals was performed, estimating attributes like Spectral Centroid and Bandwidth, which provide measures of the spectral “center of mass” and the distribution of energy across frequencies. Spectral Flatness, Rolloff, as well as measures of Skewness and Kurtosis, describe the spectral shape and distribution properties. Finally, Spectral Entropy and Peak Frequency offer insights into the randomness and dominant frequencies of the spectrum, completing a comprehensive assessment of both waveform and spectral features of the proximity signals related to mandibular movements. Hjorth parameters such as Activity, Mobility, and Complexity were computed to gauge the signal's propensity for change and irregularity, reflecting the intricacies of its pattern.

To further assess the complexity of the signal pattern within each frame, the Fractal dimension was calculated using Higuchi's method. Given that most of the signal spectrum ranged between 0.5 and 6 Hz, focus was on identifying frequency ranges that could differentiate speech from other mandible-related activities. Continuous Wavelet Transform was employed. Due to the non-stationary or transient nature of the data, and required detailed analysis, Continuous over Discrete Wavelet Transform was selected. The Morlet wavelet (“amor”) was used to decompose the signal frames into six frequency bands: 0.5-1 Hz, 1-2 Hz, 2-3 Hz, 3-4 Hz, 4-5 Hz, and 5-6 Hz. Subsequently, the statistics of each frequency band were analyzed, including metrics such as Mean Value, Standard Deviation, Energy, Entropy, Skewness, Kurtosis, Peak Frequency, Zero-Crossing Rate, and Band-power. Finally, the Cross-band dynamics (correlation with the next band) were computed for each pair of bands, to evaluate their interactions.

Considering the frequency characteristics of the dataset, two window sizes were evaluated for feature extraction: 0.5 s and 1 s. This selection was based on the understanding that a smaller window size may not adequately capture the low-frequency elements, while a larger window size could impede real-time implementation where speed is paramount. To enhance temporal resolution and mitigate boundary effects, a window overlap of 75% was implemented. Despite acknowledging the increased computational demands associated with heightened time resolution, the study prioritized resolution at this point of the analysis. Features were extracted for each subject and for each proximity channel (front, anterior, posterior, and inferior) in both ears (left and right). This yielded a total of 8 feature datasets per subject, with each dataset corresponding to a specific proxy channel.

### Optimization of Feature Subspace

Given the exploratory nature of the feature extraction process and the scarcity of literature on pertinent factors within proximity data related to mandibular motion, series of steps were conducted for feature subspace optimization, to identify the most discriminative features, and to prevent overfitting.

The optimization of the feature subspace was executed in two sequential steps: feature refinement and feature selection. In the feature refinement phase, the exploration of the associations between the features and the speech and non-speech groups was undertaken through correlation analysis, t-tests, and Least Absolute Shrinkage and Selection Operator (LASSO) regularization. Subsequently, in the feature selection phase, the Minimum Redundancy Maximum Relevance (MRMR) filter method was employed to identify the most relevant subset of features for retention.

### Feature Refinement

Upon initial assessment, it was observed that the Maximum Autocorrelation peak did not provide informative value and was consequently excluded from the feature set. In the analysis of wavelet features, the Cross-band dynamics were retained for all bands except the 5-6 Hz band, where no subsequent band existed. Conversely, due to computational challenges likely stemming from window size constraints and wavelet scale matching, all other features for the initial five bands were omitted. In the 5-6 Hz band, all features were retained except for the zero-crossing rate. This refinement process resulted in a selection of total of 48 features for further analysis.

The next step involved standardizing the data by centering each feature around its mean and scaling it by the standard deviation. Correlation analysis was then conducted between each feature and the target variable. Additionally, a t-test was performed between the speech and non-speech groups for each feature to identify statistically significant differences, with features exceeding a p-value threshold of 0.05 being removed from the dataset.

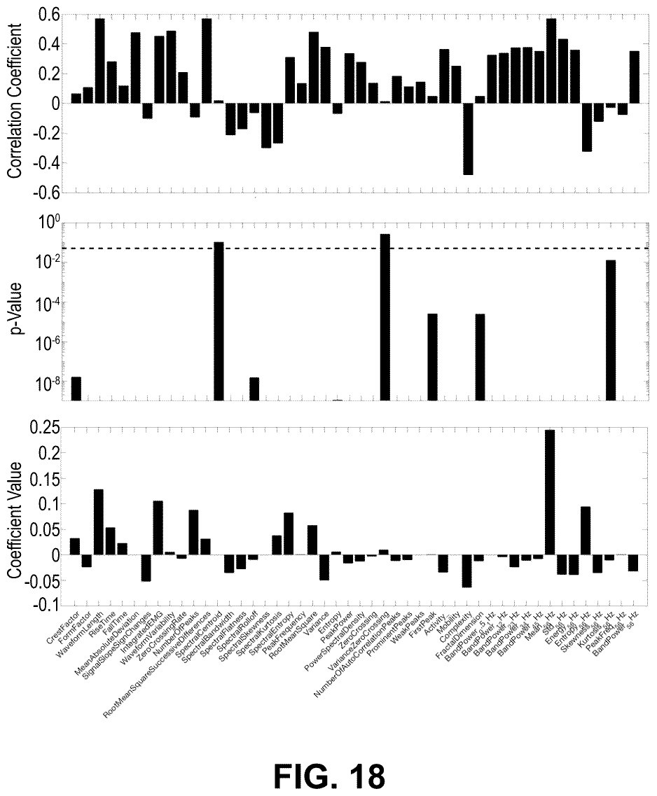

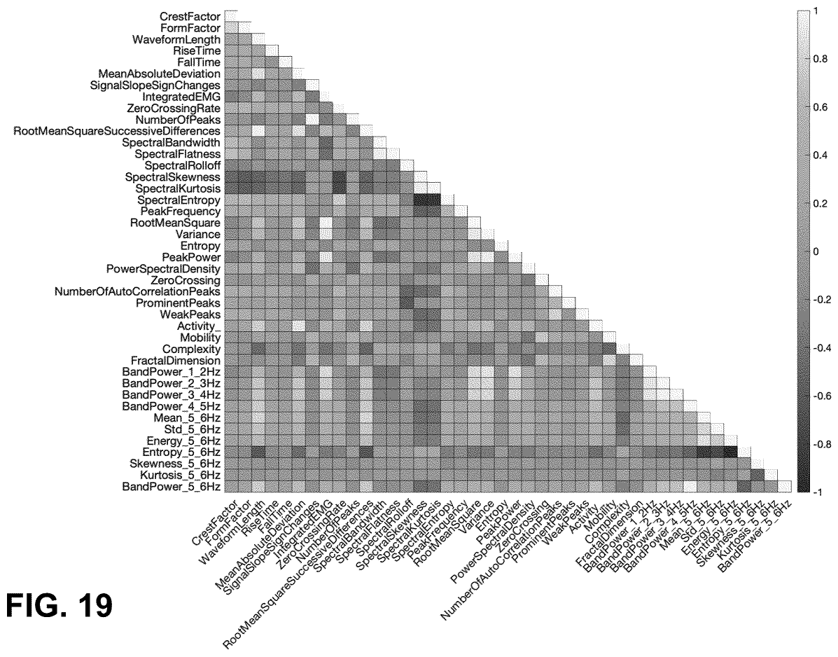

Finally, the LASSO regularization technique was applied, introducing an L1 penalty term to promote sparsity in the coefficient vector by driving some coefficients to zero. Only the features with non-zero coefficients are considered relevant by the model. This helps prevent overfitting, where the model captures noise rather than underlying patterns in the data. An illustrative example of these sequential steps is shown in FIG. 18 for subject S1 (Caucasian, female) and channel front in the right ear. For this example, window size was set to 0.5 s. To explore potential relationships among the features, a correlation plot was generated (FIG. 19).

FIG. 18 depicts feature refinement analysis for S1 and R_front (right ear canal, front sensor orientation) channel: the top panel shows correlation analysis illustrating the strength of the linear relationship between the features and the target; the middle panel shows significance levels (p-values) from the t-test conducted between the features and the target. Features surpassing the threshold denoted by the red bar (p=0.05) are not considered statistically significant. The bottom panel shows LASSO coefficients obtained with the optimal λ value, where features with zero coefficients are considered as non-relevant by the model.

FIG. 19 depicts correlations between features after feature refinement, for subject S1 and front channel of the right ear canal.

### Feature Selection

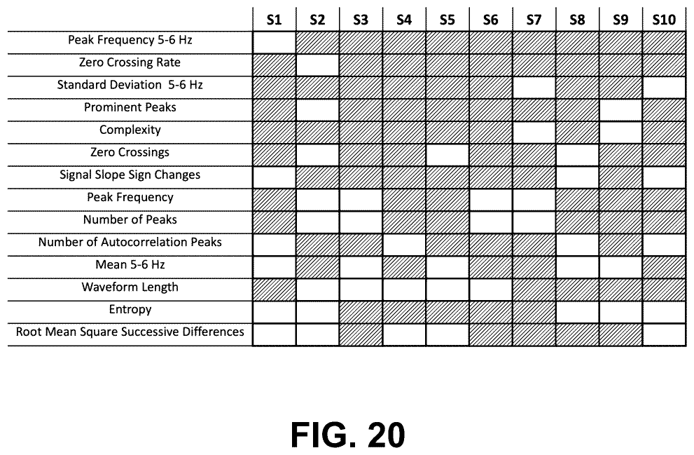

The feature dataset obtained for each subject and proximity channel underwent additional processing using the MRMR algorithm to identify the most significant features specific for each channel and subject. Following the extraction of the top 15 features for each subject, a pattern emerged where certain features were consistently prominent across multiple subjects. FIG. 20 shows recurrent features across subjects for channel front channel of the right ear canal obtained by using Minimum Redundancy Maximum Relevance feature selection algorithm. Shaded squares represent prominent features for each subject.

FIG. 20 illustrates the predominant features and their distribution among the subjects, for the channel R_front. A feature was considered recurrent if observed in at least 50% of the subjects. Based on this criterion, a total of 14 features were identified as pertinent across diverse subjects, potentially serving as globally relevant indicators for detecting speech sequences in proximity data. Notably, features such as Zero Crossing Rate and Peak Frequency within the 5-6 Hz range were present in the top 15 features of 9 out of 10 subjects, suggesting that the frequency content of proximity data may offer more insights into speech detection compared to waveform characteristics and magnitude of motion.

Training a Model to Detect Speech Relative to Baseline Activity with Inherent Movements

During the model training phase, an investigation was conducted to assess the impact of window size, number of features, and channel selection on the accuracy of speech detection. Following the optimization of the feature subset for each subject, a slight imbalance was observed in the dataset. To rectify this and ensure unbiased training, the Synthetic Minority Oversampling Technique (SMOTE) was implemented. Subsequently, individual Decision Trees models were trained for each subject. This approach was chosen due to the subject-specific nature of lower jaw movements during speech, particularly in cases where English is not the native language. Decision Trees were selected based on their superior performance compared to other classifiers (k-Nearest Neighbours, Support Vector Machine, Neural Networks) during the initial testing phase. Specifically, Boosted Trees using Ada boost was selected as an ensemble method. Maximum number of splits was set to 20, while number of learners was set to 30. Learning rate was set to 0.1.

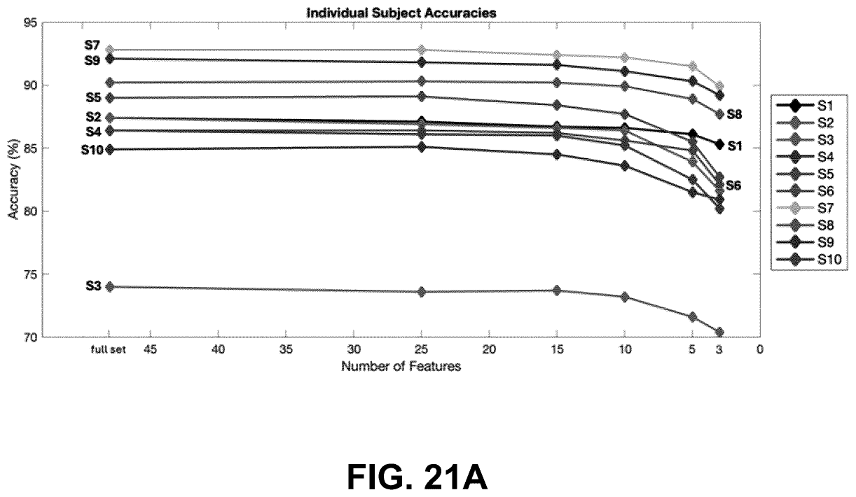

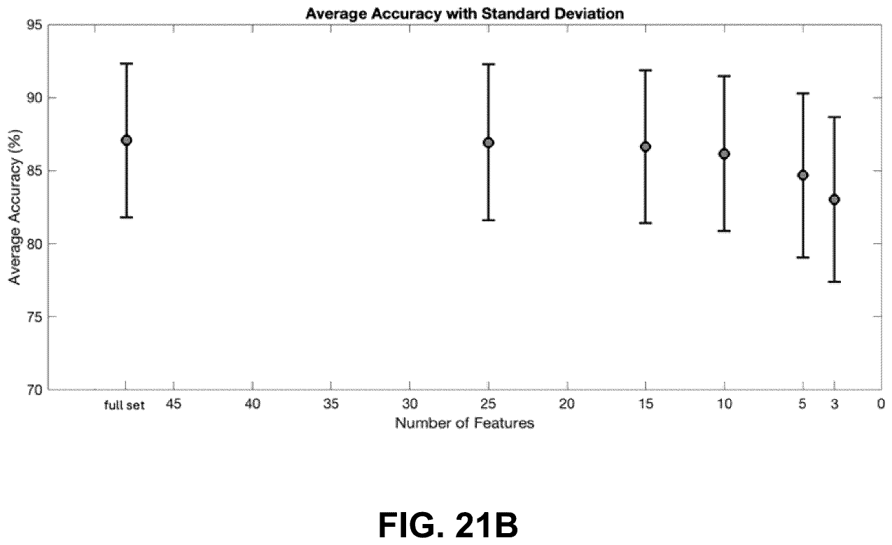

FIG. 21A and FIG. 21B show performance evaluation of the Boosted Trees classifier, designed to detect speech relative to baseline activity with inherent movements, with a window size of 0.5 s for the front channel of the right ear canal using 10-fold cross-validation. FIG. 21A shows accuracy curves illustrating the relationship between the number of features and accuracy for each subject. FIG. 21B shows average accuracy with standard deviation across 10 subjects with respect to the number of features.

The testing also indicated that a window size of 0.5 s outperformed 1 s. Regarding channel selection, the R_front channel exhibited the most significant change and provided the cleanest data. For computational efficiency, the decision was made to proceed with this channel in this phase. The training process involved 10-fold cross-validation for each subject, utilizing the complete dataset for that subject (prior to MRMR), and top 25, 15, 10, 5, and 3 features selected by MRMR. The accuracy curve, depicting the relationship between the number of features and accuracy for each subject, is presented in FIG. 21A, while FIG. 21B illustrates the average accuracy across all 10 subjects with standard deviation relative to the number of features.

Following the training of individual models, the experiment progressed towards developing a more generalized model using data pooled from multiple subjects. The initial approach was to implement a Leave-One-Patient-Out (LOPO) validation strategy, where each subject was sequentially excluded from the training dataset and used as the test subject.

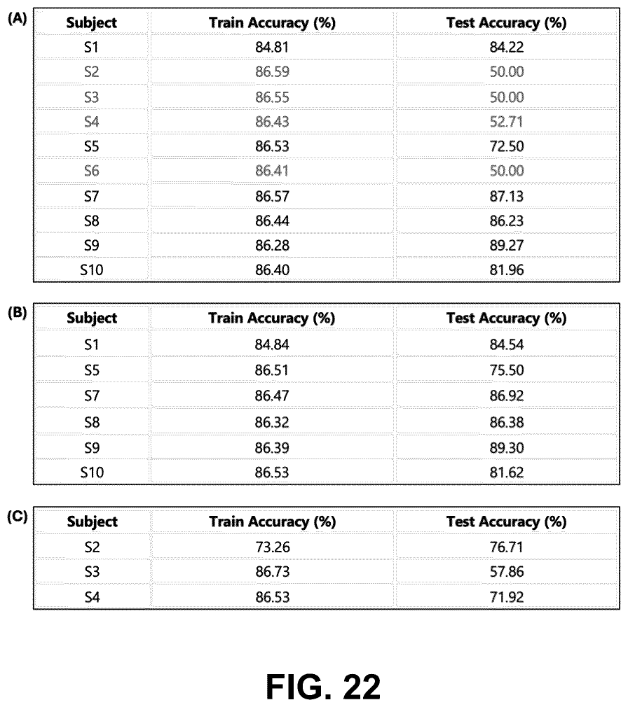

FIG. 22 depicts results of Leave-One-Patient-Out (LOPO) validation for the Boosted Trees classifier with a window size of 0.5 s for the R_front channel: Panel A shows training and test accuracy for each iteration where a specific subject is omitted from the training set and utilized for testing. Panel B shows accuracy outcomes for subgroup 1, post-exclusion of subjects S2, S3, S4, and S6. Panel C shows findings for subgroup 2, highlighting a significant enhancement in test accuracy, notably for subjects S2 and S4. Subject S6 was omitted from further subgroup analysis due to its distinct characteristics.

The findings, as shown in FIG. 22 (Panel A), showed that subjects S1, S5, S7, S8, S9, and S10 not only matched but occasionally exceeded the training accuracy in their test performances, suggesting promising results. However, subjects S2, S3, S4, and S6 showed less favorable outcomes.

This variance in performance led to a hypothesis that the characteristics of subjects S2, S3, S4, and S6 might differ significantly from those in the first subgroup. To explore this, the LOPO validation was segmented into two parts to evaluate each subgroup independently. The outcomes for the first subgroup are shown FIG. 22 (Panel B), aligning with the initial results in FIG. 22 (Panel A). The results for the second subgroup, shown in FIG. 22 (Panel C), highlighted an improvement in test accuracy, particularly for subjects S2 and S4.

Regarding subject S6, it is important to clarify that this subject was not entirely excluded from the analysis. Instead, the data from S6 did not convincingly align with either subgroup during the LOPO validation process. This decision to exclude S6 from further subgroup analysis does not suggest a dismissal of its data but indicates an absence of a clear pattern aligning with the existing subgroups. This could be attributed to unique physiological differences inherent to S6, which might have been more discernible or categorized differently if the sample size were larger. Thus, the potential for grouping subject S6 with other similar subjects in future studies remains, underscoring the need for broader data collection to capture the full spectrum of variability across individuals.

### Training a Model to Detect Speech Among Other Orofacial Activities

Upon successfully identifying isolated speech activities, the research focus expanded to include real-life scenarios where lower jaw movements are common, such as smiling, laughing, eating, yawning, and kissing. These activities represent potential sources of false triggers for the system, making it crucial to analyze and distinguish their characteristics from those of speech. Consequently, a detailed investigation was initialized to differentiate the distinct “signature” of speech observed in the outer ear canal from these non-speech activities. This comprehensive analysis incorporated all integrated mandibular movements, aiming to enhance the system's accuracy by effectively identifying and mitigating false activations.

Prior to feature extraction and model training, the study's preliminary objective was to investigate the interactions and potential groupings among various activities, guided by their statistical properties. To facilitate this analysis, several key statistical parameters were extracted for comparison: Maximum Peak Amplitude, Mean Peak Amplitude, RMS Amplitude, Energy, Dominant Frequency, Spectral Entropy, Skewness, Kurtosis, Zero Crossing Rate, and Variance. For each activity, the dataset included data from 10 events per subject, derived from performing each activity five times across two trials.

The statistical data was standardized for Principal Component Analysis (PCA, revealing that the first three principal components captured approximately 97% of the variance in data. These components were utilized as inputs for the k-means clustering algorithm. Following an exploratory analysis, three clusters were assumed, and their centroids were calculated for each subject.

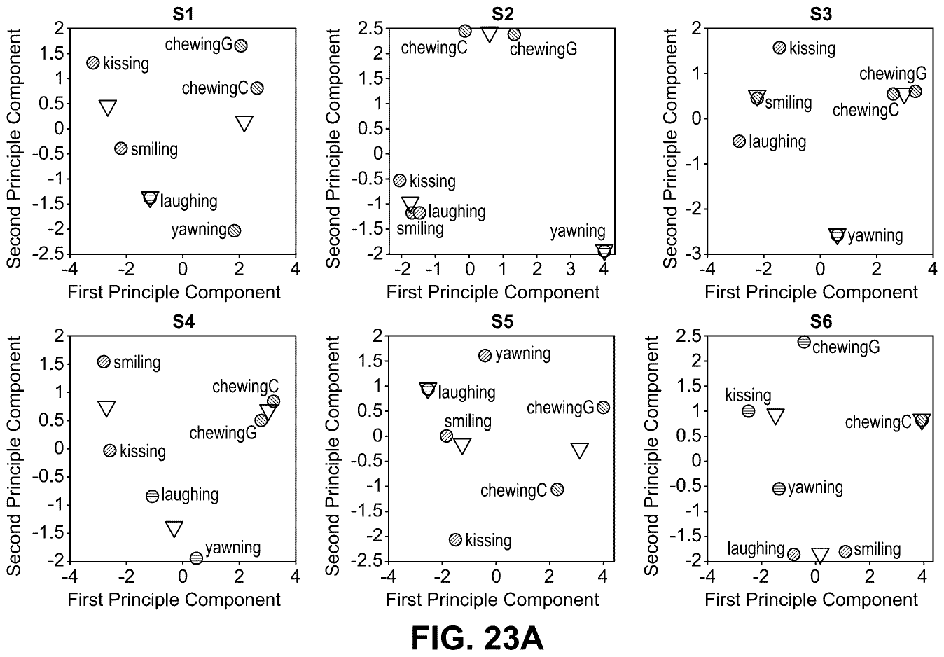

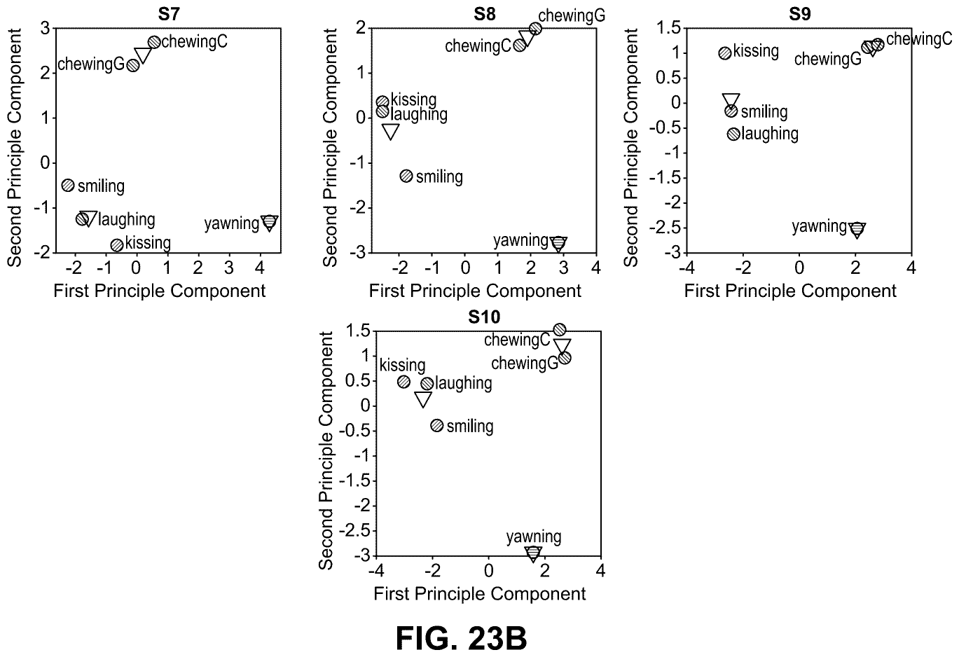

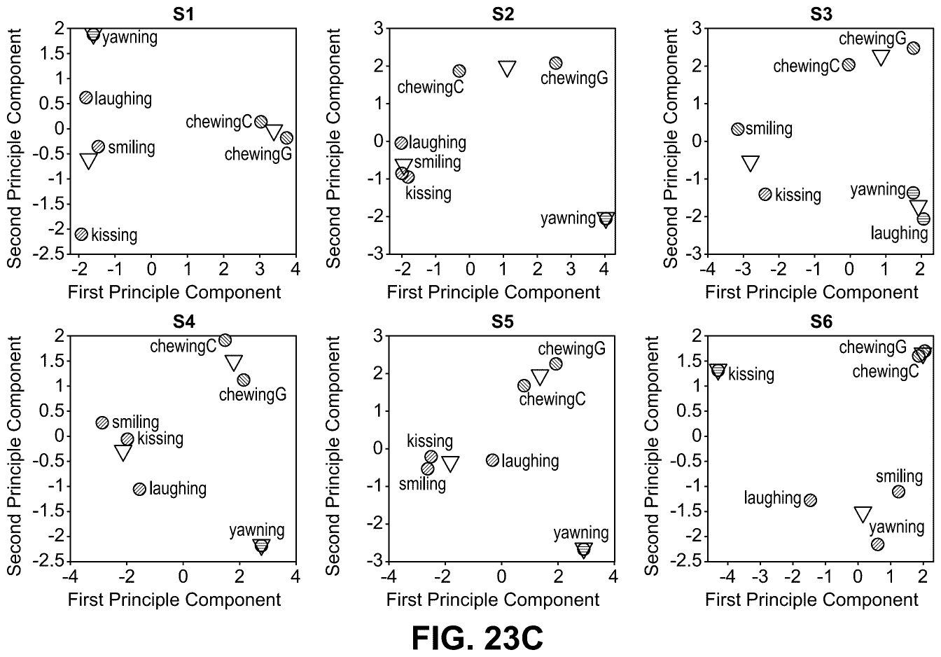

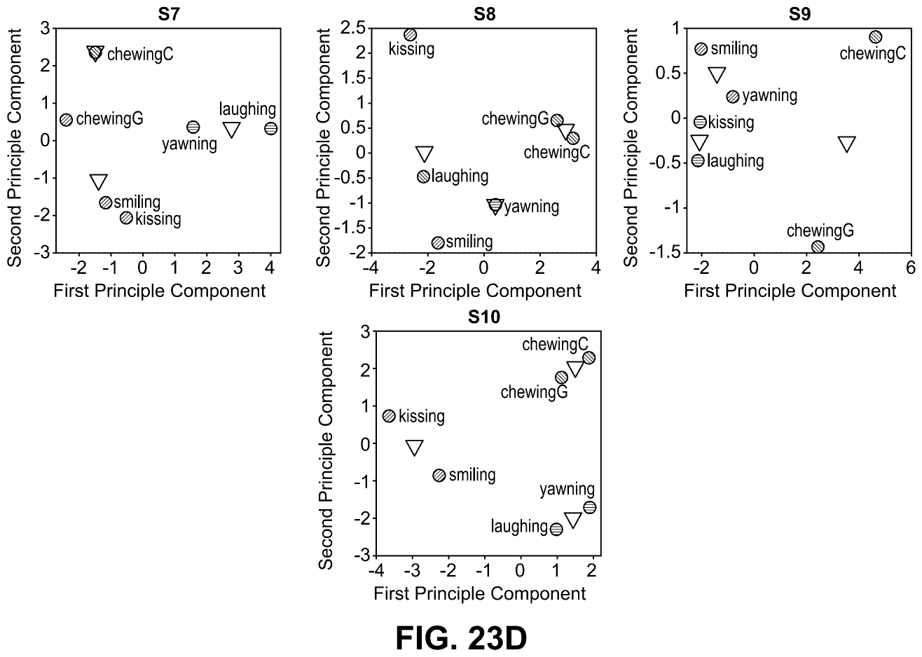

FIG. 23A, FIG. 23B, FIG. 23C and FIG. 23D show clusters of integrated mandibular movements, obtained through the k-means clustering for right (FIG. 23A and FIG. 23B) and left (FIG. 23C and FIG. 23D) ear canal. Centroids of the clusters are shown as triangles while members of the same cluster are represented with the same applied texture.

The outcomes, depicted in FIG. 23A, FIG. 23B, FIG. 23C and FIG. 23D for data collected from both the left and right ear canals, revealed distinct clustering patterns. Notably, chewing cracker and chewing gum tended to cluster together, separate from other activities. Yawning formed a distinct cluster in most subjects, while kissing, smiling, laughing, and coughing clustered together in most subjects. Yawning occasionally clustered with laughing or smiling in specific instances.

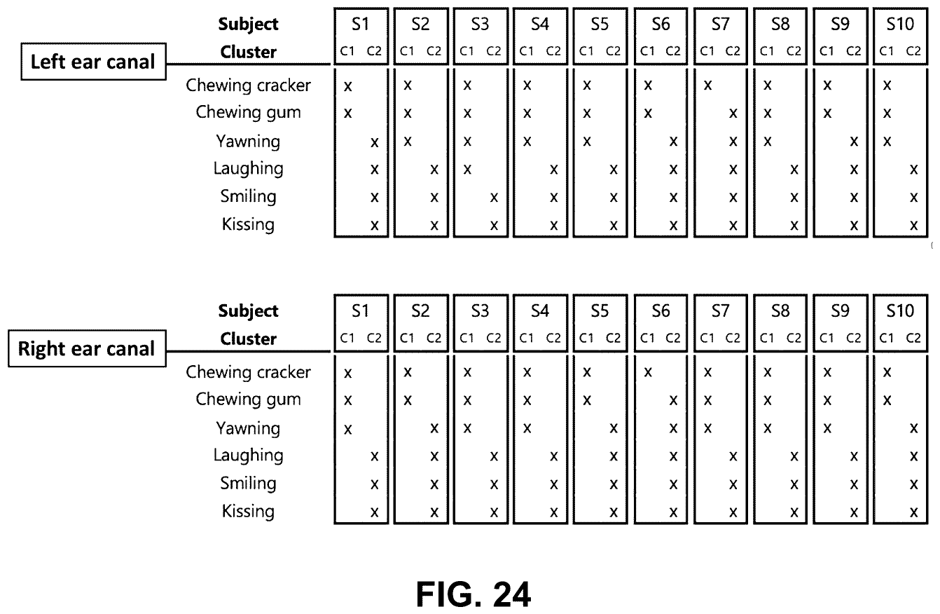

To simplify classification and model development, k-means clustering was also performed assuming two clusters to establish broader categories for classification. FIG. 24 depicts the outcomes of k-means clustering for the left (upper panel) and right (lower panel) ear canal delineated diverse clustering patterns across activities, facilitating the discernment and categorization of distinct movement types. Among 20 instances (10 subjects, 2 ear canals each), the yawning cluster was linked with the chewing cluster in 11 cases. In 6 instances, yawning was associated with the kissing, smiling, and laughing cluster. Furthermore, chewing crackers formed a distinct cluster in 2 cases, while the remaining activities were grouped together in another cluster. Notably, in a singular case, both yawning and laughing clustered with chewing, while an alternative cluster comprised smiling and kissing activities.

Following the establishment of clusters for all subjects in both ear canals, feature extraction was performed as outlined above. In addition to the features detailed above, the Harmonic-to-Noise Ratio, a metric utilized to quantify the harmonic components relative to noise in the signal was included. This inclusion was motivated by the multi-class analysis between activities such as chewing (exhibiting rhythmic patterns) and speech (lacking such periodicity). The Harmonic-to-Noise Ratio was deemed potentially informative, prompting an analysis of its magnitude and phase components. In total, 50 features were considered for classification. Given that the boosted trees (ADA boost) initially employed for speech vs. non-speech classification were designed for binary classification and not inherently suited for multi-class classification, alternative classifiers were explored.

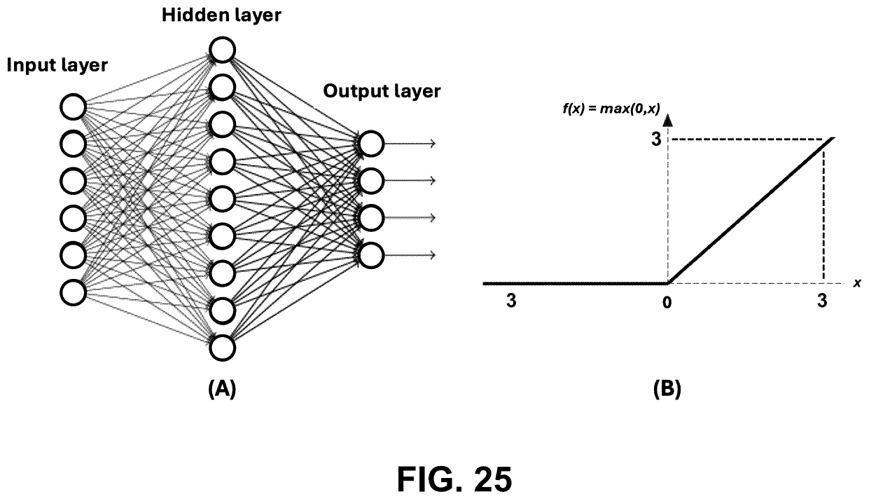

The options considered included Bagged Trees, Cubic SVM, Fine kNN, and Wide Neural Network, all of which demonstrated superior performance compared to other classifiers including the variations of Decision Trees, SVM, and kNN. Ultimately, the Wide Neural Network as shown in FIG. 25 (Panel A) exhibited the most promising performance, leading to its selection for further analysis. The neural network configuration selected featured a single fully connected hidden layer with 100 neurons and utilized the Rectified Linear Unit (ReLU) activation function as shown in FIG. 25 (Panel B). The decision to opt for a single layer was influenced by the observed overfitting and marginally inferior outcomes associated with additional layers, making them unnecessary for the complexity of the task. The ReLU activation function was selected due to its ability to mitigate issues like the Vanishing Gradient Problem encountered with sigmoid and Tan H functions.

The model was applied in two scenarios: 1) utilizing all 50 features and 2) focusing on the top 25 features selected by the MRMR algorithm for both the right and left ear canals. Performance evaluation was conducted through training models using 10-fold cross-validation and assessing F1 scores for each class.

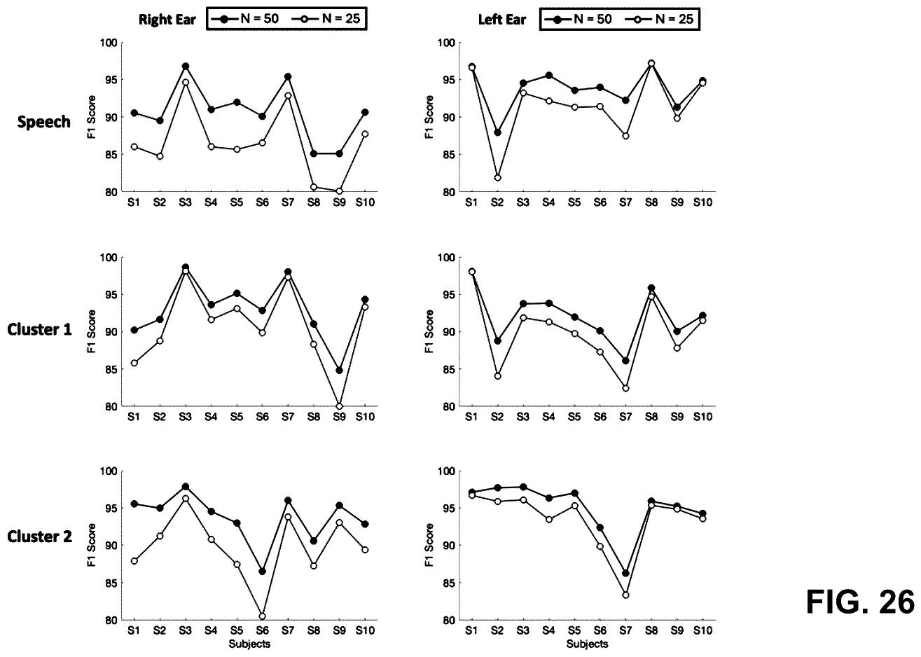

FIG. 26 depicts the results of evaluation of wide neural networks performance. The F1-scores (%) from 10-fold cross-validation are shown for individual subjects and both ear canals across N=50 and N=25 features. The results are presented for all 10 subjects for right and for left ear canal.

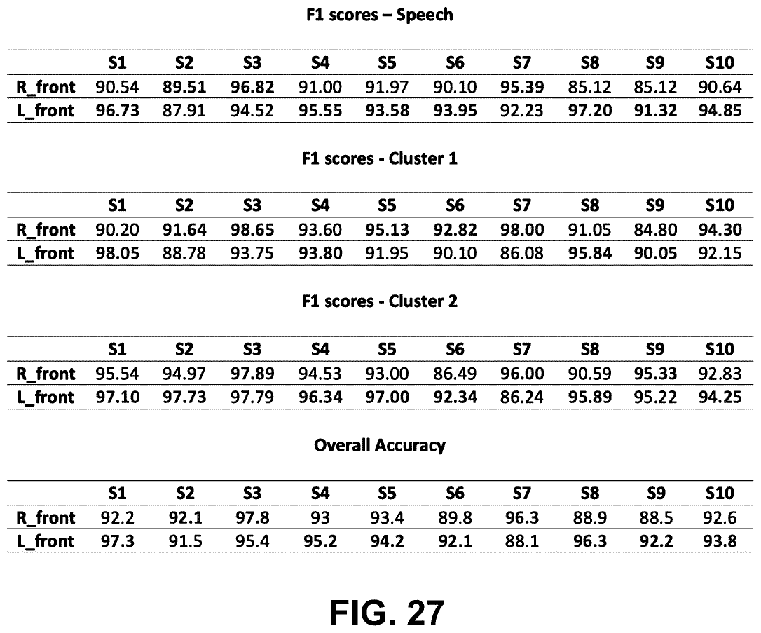

For each subject, it is evident that one ear canal outperforms the other, demonstrating higher F1 scores across the three clusters (Speech, Cluster 1, and Cluster 2) and improved overall accuracy. These findings are detailed in FIG. 27, showcasing the classification scenario utilizing all 50 features.

FIG. 27 shows the F-1 scores (%) and overall accuracy (%) of wide neural networks utilizing 50 features are expressed in percentage for the three clusters (Speech, Cluster 1, and Cluster 2) for each subject. The performance variation between the two ear canals is noticeable across all subjects.

Upon computing average statistics across the subjects, it has been shown that the Wide Neural Networks can identify speech with an average F1 score of 94.49±2.36%, Cluster 1 activities with 94.83±2.73%, and Cluster 2 activities with 95.99±1.61%. The average overall accuracy was calculated at 94.73±2.06%.

To further confirm the differences among subjects and between the left and right ear canals within the same subject, a correlation analysis was conducted on the first three principal components of the data.

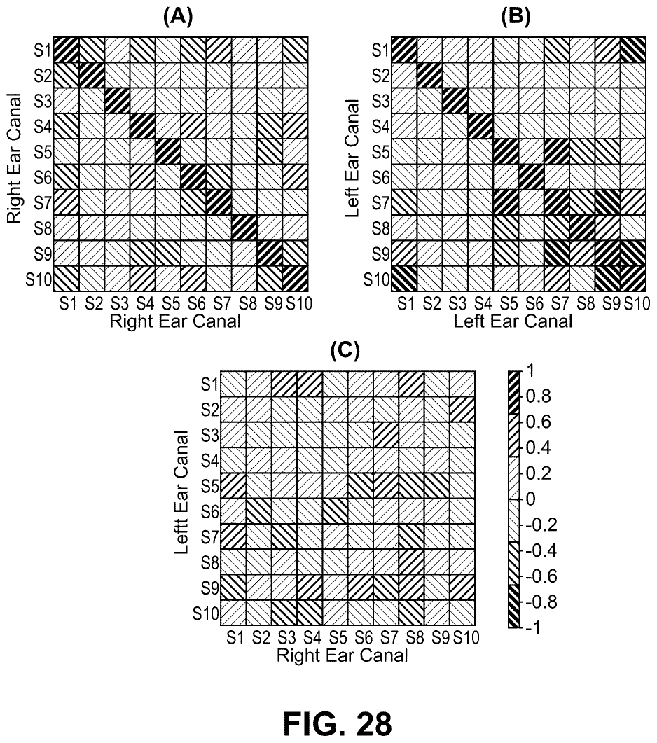

FIG. 28 shows the results of correlation analysis of the first three principal components in the data for the right ear canal (Panel A), the left ear canal (Panel B), and the comparison between the left (vertical axis) and right ear (horizontal axis) canal of the subjects (Panel C). Connections exceeding 0.7 are denoted with the corresponding numerical values.

In general, the analysis shown in FIG. 28 reveals minimal strong correlations among subjects, as indicated by correlation coefficients exceeding 0.7. In the right ear canal, there are no correlations exceeding 0.7 (Panel A). In the left ear canal, notable exceptions include positive correlations of approximately 0.7 between S5 and S7, negative correlations of similar magnitude between subjects S7 and S9, and the strongest negative correlation of approximately 0.83 observed between subjects S1 and S10 (Panel B). Furthermore, the analysis indicates that correlations between the left and right ear activity of each subject do not appear to be strong, which could explain the performance variation between the two ear canals across the subjects as shown in FIG. 28 (Panel C).

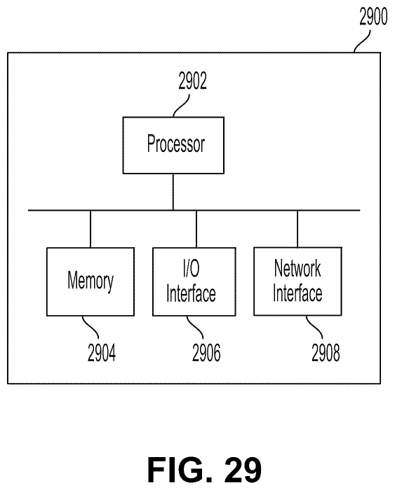

FIG. 29 is a schematic diagram of computing device 2900 which may be used to implement processing device 110.

As depicted, computing device 2900 includes at least one processor 2902, memory 2904, at least one I/O interface 2906, and at least one network interface 2908.

Each processor 2902 may be, for example, any type of general-purpose microprocessor or microcontroller, a digital signal processing (DSP) processor, an integrated circuit, a field programmable gate array (FPGA), a reconfigurable processor, a programmable read-only memory (PROM), or any combination thereof.

Memory 2904 may include a suitable combination of any type of computer memory that is located either internally or externally such as, for example, random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like.

Each I/O interface 2906 enables computing device 2900 to interconnect with one or more input devices, such as a keyboard, mouse, camera, touch screen and a microphone, or with one or more output devices such as a display screen and a speaker.

Each network interface 2908 enables computing device 2900 to communicate with other components, to exchange data with other components, to access and connect to network resources, to serve applications, and perform other computing applications by connecting to a network (or multiple networks) capable of carrying data including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these.

For simplicity only, one computing device 2900 is shown but processing device 110 may include multiple computing devices 2900. The computing devices 2900 may be the same or different types of devices. The computing devices 2900 may be connected in various ways including directly coupled, indirectly coupled via a network, and distributed over a wide geographic area and connected via a network (which may be referred to as “cloud computing”).

For example, a computing device 2900 may be a server, network appliance, set-top box, embedded device, computer expansion module, personal computer, laptop, personal data assistant, cellular telephone, smartphone device, UMPC tablets, video display terminal, gaming console, or any other computing device capable of being configured to carry out the methods described herein.

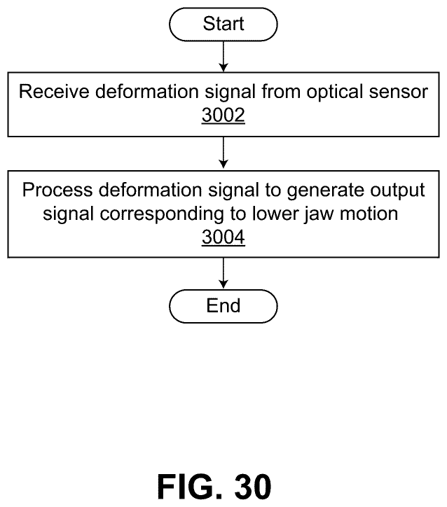

FIG. 30 is a flowchart illustrating a method that may be performed at monitoring system 100, in accordance with an embodiment. At step 3002, monitoring system 100 receives a deformation signal from optical sensor installed on an in-ear device; at step 3003, monitoring system 100 processes the deformation signal to generate output signal corresponding to lower jaw motion.

In an example use case, monitoring system 100 and in-ear device(s) 102 are configured to sense mandibular motion manifested as volumetric or linear deformation of the cartilaginous segment of the external ear canal during jaw movements. A particular application may be when a user performs jaw movements associated with speech articulation (e.g., protrusion, retraction, elevation/depression, lateral excursion and integrated articulatory sequences), the anterior and frontal canal walls deform, modulating the optical return captured by integrated proximity sensors. An in-ear device 102 samples these changes as time-series proximity data within a bandwidth corresponding to lower jaw kinematics and orofacial dynamics. In some embodiments, multi-axis sensing within the ear canal is employed (e.g., anterior, frontal, inferior, posterior orientations) to improve sensitivity to speech-related deformation while enabling discrimination from non-speech activities such as mastication, coughing, yawning, or head motion.

Monitoring system 100 processes the proximity signals locally (e.g., at in-ear device 102 or at processing device 110) and/or remotely to derive activity indicators and control states. Preprocessing can include band-limiting and normalization suitable for low-frequency biomechanical signals, followed by feature extraction in the time and/or frequency domains. A classifier, rule engine, or neural network may be configured to distinguish speech intention from other mandibular or orofacial activities based on extracted features, temporal envelopes, onset/offset timings, and inter-sensor patterns. In some use cases, monitoring system 100 detects speech onsets that precede audible phonation and generates control tokens reflecting at least one of: speech present/absent, onset/termination timestamps, estimated articulation intensity, phoneme- or word-level segmentation cues, or confidence scores.

Upon detection of a speech intention state, monitoring system 100 may generate and output a control signal for downstream systems. In one embodiment, a wireless link transmits a low-latency trigger to a sound transducer, such as an electrolarynx or other external voice prosthesis, to initiate and modulate artificial voice generation without manual actuation. In another embodiment, the control signal modulates transducer parameters in real time, including amplitude, voicing state, and pitch contour, using the dynamics of the proximity signal as an input envelope. The system may further implement safety and debouncing logic to suppress false activations during non-speech activities and to maintain stable operation in mobile, real-world conditions.

Beyond voice restoration, the detected mandibular activity stream can be utilized for multiple downstream applications. Exemplary use cases include hands-free human-machine interfaces, silent speech input in high-noise or privacy-sensitive environments, assistive communication for users with impaired phonation, and context-aware control of wearable devices such as a smart watch. In certain embodiments, historical proximity data is used to personalize classifier parameters to an individual's ear canal morphology and motion signatures, improving specificity and sensitivity over time.

In some embodiments, the above use cases may enable convenient, discreet, socially acceptable, and/or robust control of downstream activations and related interfaces by leveraging in-ear sensing of jaw-induced ear canal deformation.

The foregoing discussion provides many example embodiments of the inventive subject matter. Although each embodiment represents a single combination of inventive elements, the inventive subject matter is considered to include all possible combinations of the disclosed elements. Thus, if one embodiment comprises elements A, B, and C, and a second embodiment comprises elements B and D, then the inventive subject matter is also considered to include other remaining combinations of A, B, C, or D, even if not explicitly disclosed.

The embodiments of the devices, systems and methods described herein may be implemented in a combination of both hardware and software. These embodiments may be implemented on programmable computers, each computer including at least one processor, a data storage system (including volatile memory or non-volatile memory or other data storage elements or a combination thereof), and at least one communication interface.

Program code is applied to input data to perform the functions described herein and to generate output information. The output information is applied to one or more output devices. In some embodiments, the communication interface may be a network communication interface. In embodiments in which elements may be combined, the communication interface may be a software communication interface, such as those for inter-process communication. In still other embodiments, there may be a combination of communication interfaces implemented as hardware, software, and combination thereof.

Throughout the foregoing discussion, numerous references will be made regarding servers, services, interfaces, portals, platforms, or other systems formed from computing devices. It should be appreciated that the use of such terms is deemed to represent one or more computing devices having at least one processor configured to execute software instructions stored on a computer readable tangible, non-transitory medium. For example, a server can include one or more computers operating as a web server, database server, or other type of computer server in a manner to fulfill described roles, responsibilities, or functions.

The technical solution of embodiments may be in the form of a software product. The software product may be stored in a non-volatile or non-transitory storage medium, which may be a compact disk read-only memory (CD-ROM), a USB flash disk, or a removable hard disk. The software product includes a number of instructions that enable a computer device (personal computer, server, or network device) to execute the methods provided by the embodiments.

The embodiments described herein are implemented by physical computer hardware, including computing devices, servers, receivers, transmitters, processors, memory, displays, and networks. The embodiments described herein provide useful physical machines and particularly configured computer hardware arrangements.

Of course, the above-described embodiments are intended to be illustrative only and in no way limiting. The described embodiments are susceptible to many modifications of form, arrangement of parts, details and order of operation. The disclosure is intended to encompass all such modification within its scope, as defined by the claims.

What is claimed is:

1. A system for monitoring lower jaw motion, the system including:
an insert for insertion into an ear canal of a user, the insert including a plurality of optical sensors, each for sensing deformation of a corresponding region of the ear canal caused by a lower jaw motion; and
one or more processors and one or more memories coupled with the one or more processors, the processors and memories configured to:
receive a deformation signal from at least one of the optical sensors, the deformation signal indicative of deformation of the corresponding region of the ear canal; and
process the deformation signal to generate an output signal corresponding to the lower jaw motion.

2. The system of claim 1, wherein the plurality of optical sensors includes an infrared proximity sensor.

3. The system of claim 1, wherein the plurality of optical sensors includes an optical sensor aligned to sense deformation of an anterior wall of the ear canal.

4. The system of claim 1, wherein the plurality of optical sensors includes an optical sensor aligned to sense deformation of a posterior wall of the ear canal.

5. The system of claim 1, wherein the plurality of optical sensors includes an optical sensor aligned to sense deformation of an inferior cartilaginous wall of the ear canal.

6. The system of claim 1, wherein the plurality of optical sensors includes an optical sensor aligned towards an eardrum.

7. The system of claim 1, further including a wireless transceiver to transmit the deformation signal wirelessly from the at least one of the optical sensors.

8. The system of claim 1, wherein the insert is a first insert for insertion into a first ear canal of the user, and the system further includes:
a second insert for insertion into a second ear canal of the user, the second insert including a further plurality of optical sensors, each for sensing deformation of a corresponding region of the second ear canal caused by lower jaw motion.

9. The system of claim 1, wherein the output signal encodes a particular user intent.

10. The system of claim 9, wherein the particular user intent includes at least one of a speech intent, a chewing intent, or a swallowing intent.

11. The system of claim 1, wherein the output signal encodes a particular user activity, request, or command.

12. The system of claim 11, wherein the particular user activity includes at least one of a particular unit of speech, a particular unit of chewing, a particular unit of smiling, a particular unit of laughing, a particular unit of yawning, or a particular unit of swallowing.

13. The system of claim 11, where in the particular user activity includes a particular kissing.

14. The system of claim 1, wherein the output signal is generated by applying a machine learning model to the deformation signal.

15. An in-ear device for monitoring lower jaw motion, the device including:
an insert for insertion into an ear canal, the insert including a plurality of optical sensors, each for sensing deformation of a corresponding region of the ear canal caused by a lower jaw motion; and
an output interface for transmitting a deformation signal sensed by at least one of the optical sensors, the deformation signal indicative of deformation of the corresponding region of the ear canal.

16. The in-ear device of claim 15, wherein the plurality of optical sensors includes an infrared proximity sensor.

17. The in-ear device of claim 15, wherein the plurality of optical sensors includes an optical sensor aligned to sense deformation of an anterior wall of the ear canal, a posterior wall of the ear canal, or an inferior cartilaginous wall of the ear canal.

18. The in-ear device of claim 15 wherein the plurality of optical sensors includes an optical sensor aligned towards an eardrum.

19. The in-ear device of claim 15, wherein the output interface includes a wireless transceiver to transmit the deformation signal wirelessly from the at least one of the optical sensors.

20. A method for monitoring lower jaw motion including:
receiving a deformation signal from at least one optical sensor for sensing deformation of a corresponding region of the ear canal caused by a lower jaw motion, disposed into an ear canal of a user; and
processing the deformation signal to generate an output signal corresponding to the lower jaw motion.
